(19) United States
(12) Patent Application Publication
(10) Pub. No.: US 2011/0151031 A1
(43) Pub. Date: Jun. 23, 2011

(54) COMPOSITIONS AND METHODS FOR UTILIZING THE SAME

(75) Inventor: Yoshihiro Futamura, Aichi (JP)

(73) Assignee: DBC, LLC, Lehi, UT (US)

(21) Appl. No.: 13/059,889

(22) PCT Filed: Aug. 18, 2009
(86) PCT No.: PCT/US2009/054190

§ 371 Date: Feb. 18, 2011

Related U.S. Application Data
(60) Provisional application No. 61/090,170, filed on Aug. 19, 2008.
(60) Provisional application No. 61/090,166, filed on Aug. 19, 2008.
(60) Provisional application No. 61/090,167, filed on Aug. 19, 2008.

(51) Int. Cl. A61K 36/15; A61K 8/96; A61K 8/49; A61K 36/00; A61P 17/00; A61P 29/00; A61Q 19/08; C12P 17/06; C12P 1/00; C12P 1/04; A23L 1/30

(52) U.S. Cl. 424/732; 514/455; 424/765; 424/735; 424/736; 424/758; 514/456; 424/725; 424/770; 424/774; 424/778; 435/125; 435/41; 435/170; 426/648

(57) ABSTRACT

Xanthone compositions are provided herein. Formulations containing such compositions and methods of using such formulations and compositions are also provided. The present description additionally sets forth processes for making one or more compositions including compositions capable of promoting collagen production, antioxidant compositions, and anti-inflammatory compositions.

### TECHNICAL FIELD

Compositions, including compositions for promoting collagen production, antioxidant compositions, and anti-inflammatory compositions, are provided herein. Products including such compositions, as well as methods for producing and using such compositions and products, are also provided.

### BACKGROUND

### Compositions for Promoting Collagen Production

Collagens are proteins that are present in skins, bones, cartilages and blood vessels as well as membranous tissues within various anatomies. There are more than 10 kinds of collagens and they function to build skeletons of parenchyma, build bones, protect tissues from inflammation, inhibit invasion of cancers, prevent metastasis of cancers, etc. Enzymes capable of breaking down collagen, such as collagenases and matrix metalloproteases, are also present in many organisms and may be responsible for the breakdown of collagen materials within a subject. For example, in arthritis, collagenases are produced by inflammatory tissues and inflammatory cells such as neutrophils produced in areas of cartilage, resulting in breakdown of collagens (See, e.g., Peel N et al., Baillieres Clin Rheumatol, 6, 351-372, 1992).

Because production and function of collagens have been associated with inhibiting cancer metastasis, inhibiting angiogenesis associated with cancer metastases, inhibiting growth of cancer cells, inhibiting arthritis, improving inflammation, inhibiting osteoporosis, inhibiting and improving various conditions of the skin, such as the appearance or formation of cellulite, wrinkles, and sagging of the skin, methods for stimulating collagen production have been the focus of various research efforts. In some instances, plants have been found to contain and produce constituents that promote collagen production, and isolation and refinement of such constituents has been explored (Niizawa A et al., Clin. Exp. Rheumatol, 21, 57-62, 2003). In another example, compositions containing phenol compound derived from mimosa purica (refer to Patent Reference 1, for example) have been described. In yet a further example, inhibitors of collagenase containing at least one of magnolol and honokiol have been reportedly isolated from magnolia obovata, (See, e.g., Japanese Patent No. 2886523). Even further, terpenes derived from plants have been reportedly used in cosmetic compositions for promoting collagen production (See, e.g., National Publication of Translated Version of Japanese Patent App. Serial No. 2002-516837).

### Antioxidant Compositions

Naturally occurring compounds having antioxidant action are known. For example, plant-derived polyphenols have been identified as having antioxidant activity. Further, naturally occurring vitamins, ubiquinones, alpha-lipoic acids and astaxanthines having antioxidant activity have been identified. Some naturally occurring compounds having antioxidant action, however, may exhibit short circulating half-lives, may be difficult to isolate, purify or formulate, may not exhibit sufficient potency for therapeutic application, or may be poorly stable.

In some instances, antioxidant action has been correlated with antimutagenic action. For instance, Japanese Patent Publication No. 1996-259453 and Japanese Patent Publication No. 2005-179373 describe compositions, including juice compositions, that exhibit mutation-suppressing activity. These references also describe methods for obtaining the polyphenols or polyphenol compositions from plant materials, such as from apple fruit or immature fruits from plants belonging to the Rosaceae family.

Japanese Patent Publication No. 2002-47196 describes polyphenols, antioxidants, antihypotensive agents, and anti-allergenic agents derived from extracts obtained from immature fruits of apple, pear or peach trees. The refined fractions described in this reference include caffeic acid derivatives, p-coumaric acid derivatives, flavan-3-ols, flavonols and dihydrochalconic acid as simple polyphenol compounds and condensed tannins, which are regular polymers of catechins, as high molecular weight polyphenol compounds.

Xanthones are polyphenol compounds that are naturally synthesized by plants and microorganisms. For example, xanthones are found in plants of the Clusiaceae family, such as the mangosteen and Hypericum perforatum. As indicated by Japanese Patent Publication No. 2006-89661, some xanthone compounds have been reported to exhibit antioxidant properties.

### Anti-Inflammatory Compositions

Inflammatory cytokines are proteins produced by cells such as leukocytes, macrophages, lymphocytes and monocytes, which induce symptoms such as fever, pain and rubor. Inflammatory cytokines induce, for example, prostaglandins and thromboxanes, which can induce symptoms such as fever, swelling, redness and pain. Examples of inflammatory cytokines include Interleukin-1 alpha, Interleukin-1 beta, Interleukin-6, Interleukin-8, and tumor necrosis factor alpha (TNF alpha). Among these, Interleukin-1 alpha and Interleukin-1 beta are released by inflammatory cells such as monocytes and lymphocytes to promote secretion of Interleukin-6, Interleukin-8, and TNF alpha. Interleukin-6 is involved in fever and inflammatory tissue disorders. TNF alpha, also known as cachectin, is released in cancers and cachexia and results in exacerbation of systemic symptoms and discomfort (See, e.g., Grignari G. et al., Haematologica, 85, 967-972, 2000). Production of inflammatory cytokines can be induced as a result of infection by, for example, bacteria, parasites or viruses. Further, it has been reported that transcription factors, such as kappa B (NF-kappa B) and NF-IL6, promote transcription of messenger RNAs of inflammatory cytokines (see, e.g., Driscoll, E. et al., Environ. Health Perspect, 105, 1159-1164, 1997).

Substances for inhibiting production of inflammatory cytokines are known. For example, corticosteroid preparations, which inhibit protein kinase C and transcription factors of inflammatory cytokines in inflammatory cells, can serve to suppress production of inflammatory cytokines (See, e.g., Roman, J. et al., Immunology, 98, 228-237, 1999). Examples of corticosteroids with anti-inflammatory properties include clobetasol propionate, diflorasone acetate, prednisolone, and cortisone. In some instances, however, corticosteroids may be associated with undesirable side effects, such as increased instances of decreased immune response, opportunistic infection, hypoadrenalism, and dermatitis, such as steroid purpura (See, e.g., Bruner, C. R. et al., Dermatol. Online J, 9, 2, 2003).

There are also reports of plant-derived substances that exhibit anti-inflammatory properties. For example, glycyrrhiza and other herbs are known to contain constituents that provide anti-inflammatory function, and in some instances these materials can be found in pharmaceutical and consumer products. Glycyrrhiza contains glycyrrhizic acid, which reportedly has steroid-like action (See, e.g., Sasaki, H. et al., Pathobiology, 70, 229-236, 2002-2003). Other publications suggest the anti-inflammatory properties of Chinese herbal extracts and preparations containing such compositions with respect to anise among such materials (See, e.g., Japanese Patent Publication No. 2005-187394) or describe anti-inflammatory foods (See, e.g., Japanese Patent Publication No. 1993-139950). In a further example, cosmetic compositions containing at least one hydroquinone glucoside and a crude drug or herbal extract having skin-lightening action have also been discussed in the art (See, e.g., Japanese Patent Publication No. 1999-318387). With respect to polyphenol compounds, Japanese Patent Publication No. 2006-219471 describes methods for metabolic inhibition of arachidonic acid using Altol as an active ingredient.

### BRIEF DESCRIPTION OF THE DRAWINGS

FIG. 1 provides an HPLC Chromatogram of the composition of Example 1 and a control composition.

FIG. 2 provides a Total Ion Chromatogram (“TIC”) of the composition of Example 1.

FIG. 3 illustrates a mass spectrum of chromatogram peak at 21.876 minutes, API-ES Positive Scan Mode.

FIG. 4 illustrates a mass spectrum of chromatogram peak at 22.081 minutes, API-ES Positive Scan Mode.

FIG. 5 illustrates a mass spectrum of chromatogram peak at 22.428 minutes, API-ES Positive Scan Mode.

FIG. 6 illustrates a mass spectrum of chromatogram peak at 47.868 minutes, API-ES Positive Scan Mode.

FIG. 7 provides an HPLC-PDA Chromatogram of the composition of Example 6 and a control composition.

FIG. 8 provides a HPLC-PDA chromatogram of the extract of Example 13 and a control composition.

### DESCRIPTION OF THE INVENTION

In one embodiment, the compositions described herein can be obtained, at least in part, from processing plant materials. In a particular embodiment, the compositions described herein are produced using plant materials containing one or more xanthone compounds. The compositions described herein can be produced using methods detailed in the present disclosure. Moreover, in further embodiments, compositions according to the present disclosure can be formulated into, for example, personal care products, pharmaceutical products, and nutritional supplements. In one such embodiment, a composition according to the present invention is formulated into a personal care formulation for topical application, wherein such formulation is administered to a subject. In particular embodiments, the compositions described herein exhibit one or more physical and/or analytical characteristics.

The compositions described herein can be derived from plant materials containing xanthone compounds. For example, a variety of plant species produce xanthone compounds which can be used in methods for producing compositions as described herein. Plant materials that may be used as sources for xanthone compounds include, but are not limited to, the leaves and stems of green tea, the seeds, fruits, roots, leaves and stems of lotus as well as the leaves, stems, blossoms, fruits or roots of Kothala Himbutu, Hercampure, Allium victorialis var. platyphyllum, onion, garlic, soybean, Rumex japonicus, glycyrrhiza, jewelweed, Helwingia japonica, barley, Pueraria lobata, capsicum, Japanese persimmon, pear, chestnut, Aralia elata, Wasabia japonica, bracken, rice, wheat, corn, radish, grape, cherry, pine, Aucuba japonica, Rubia argyi, Mallotus japonicus, Akebia quinata, Gynostemma pentaphyllum, Polygonatum odoratum, aloe, Epimedium grandiflorum var. thunbergianum, Polygonum cuspidatum, Achyranthes bidentata var. japonica, Thymus quinquecostatus, Araliaceae, Prunella vulgaris subsp. asiatica, Aralia cordata, Japanese apricot, Quercus salicina, Senna obtusifolia, coptis rhizome, Plantago asiatica, Atractylodes japonica, Abelmoschus esculentus, Hypericum erectum, Xanthium strumarium, Patrinia scabiosifolia, Glechoma hederacea subsp. grandis, Trichosanthes cucumeroides, Pinellia ternata, Chamaecrista nomame, Dianthus superbus var. longicalycinus, Heterotropa kooyana var. nipponica, Jerusalem artichoke, Platycodon grandiflorus, Catalpa ovata, Phellodendron amurense, Ajuga decumbens, Agrimonia pilosa, Veronicastrum sibiricum subsp. japonicum, Chaenomeles japonica, kudzu, common gardenia, Nuphar japonicum, Kobushi magnolia, Gleditsia japonica, soapwort, Smilax china, Cornus officinalis, Ophiopogon japonicus, Bletilla striata, Japanese honeysuckle, Japanese parsley, Swertia japonica, Magnolia salicifolia, dandelion, Imperata cylindrica, Adenophora triphylla, Farfugium japonicum, Aesculus turbinata, Panax japonicus, Nandina domestica, Rosa multiflora, Stellaria media, Coix lacryma-jobi var. ma-yuen, Gnaphalium affine, Plectranthus japonicus, Trapa japonica, Pyrrosia lingua, Japanese loquat, Petasites japonicus, Far East Amur adonis, Wisteria floribunda, Actinidia polygama, Leonurus japonicus, glutinous yam, beefsteak geranium, mugwort, gentian, Forsythia suspensa, Chimonanthus praecox, and Sanguisorba officinalis.

In one embodiment, a method for producing compositions as described herein utilizes materials, such as the leaves, barks, blossoms, fruits and roots, taken from Hypericum perforatum, Garcinia subelliptica, Clusia Rosea, Hypericum trichocaulon, Hypericum calycinum, Calophyllum inophyllum, Hypericum kalmianum, Hypericum ‘Hidcote’, and Hypericum androsaemum.

In another embodiment, a method for producing the compositions described herein utilizes whole fruit, fruit pulp, fruit pericarp, leaves, stems, blossoms and roots, from plants of the Clusiaceae family, Hercampure, or Kothala Himbutu. In one such embodiment, materials taken from Garcinia mangostana, the mangosteen, are used. The mangosteen is a tree that grows in tropical areas, such as in Southeast Asia, South America and Africa, and fruits several times a year. Where mangosteen fruit is used as a food, the food processing industry generally removes the mangosteen fruit pericarps, which are then disposed of as a waste material. However, both the fruit pulp and the fruit pericarp of the mangosteen fruit are known to contain xanthone compounds and can be used as a raw material from which the compositions described herein can be produced. In one embodiment, the xanthone compounds from the mangosteen fruit may be used in compounds for collagen promoting. In another embodiment, the xanthone compounds from the mangosteen fruit may be used as a raw material from which antioxidant compositions can be produced. In yet another embodiment, the xanthone compounds from the mangosteen fruit may be used as a raw material from which anti-inflammatory compositions can be produced. Where used in the methods described herein, the mangosteen fruit (both the fruit pulp and the pericarp) may be fresh or dried.

The plant materials used as a source of xanthone compounds in the compositions and methods described herein can be ground prior to use. Grinding of the desired plant materials, such as, for example, the whole fruit or the fruit pulp or fruit pericarp of the mangosteen fruit, can be carried out using known grinding appliances, such as a Jiyu Mill, Super Jiyu Mill, Sample Mill, Goblin, Super Clean Mill, or Micros manufactured by Nara Machinery Co., Ltd., and where needed, a commercially available vacuum dryer may also be employed, such as a compact vacuum dryer manufactured by Toyoriko Co, Ltd., a compact thermal conduction vacuum dryer DPTH-40 manufactured by Matsui Mfg. Co., Ltd., or a Cleandry VD-7 or VD-20 manufactured by AQM Kyushu Technos. Once the desired plant materials are prepared, they can be mixed with one or more additional constituents under conditions that drive production of a composition for promoting collagen production. In another embodiment, the prepared plant materials may be mixed with one or more additional constituents under conditions that drive production of an antioxidant composition. In yet another embodiment, once the desired plant materials are prepared, they can be mixed with one or more additional constituents under conditions that facilitate production of an anti-inflammatory composition.

In one embodiment, ground plant materials are combined with one or more solvents and one or more additional chemical or biologic constituents to form a reaction mixture. The one or more additional chemical or biologic constituents included in the reaction mixture are chosen to react with the plant material to produce the desired composition. In particular embodiments, the chemical constituents may include one or more organic or mineral acids. In one such embodiment, a solvent used in forming a reaction mixture may include an acidic aqueous solution, such as, for example, an acetic acid, citric acid, malic acid, succinic acid, oxalic acid, hydrochloric acid, of sulfuric acid solution, including acidic solutions including a combination of two or more such acids. In another embodiment, a solvent used in forming a reaction mixture may include an acidic aqueous solution selected from a citric acid solution, an oxalic acid solution, a malic acid solution, and an acetic acid solution. Where an acidic solution is used in a reaction mixture as described herein, the pH of such a solution may be selected from, for example, a pH ranging from about 1.5 to about 6.5. Where desired to facilitate or speed the desired chemical reactions, heat may also be applied to the reaction mixture and one or more catalysts, such as a metallic catalyst, including a magnesium or aluminum catalyst, may be used. Even further, the reaction mixture may be prepared to facilitate one or more fermentation steps useful in achieving a composition as described herein.

Where fermentation is used in a process for producing the compositions described herein, such fermentation can be carried out using standard equipment, such as fermentation vessels. Moreover, where a fermentation step is desired, a method as described herein may include creation of a reaction mixture that includes a solvent, plant material containing xanthone compounds, a fermentation base, such as soybeans, and one or more biologic constituents, such as one or more microorganisms, including, but not limited to, Lactobacillus species, including Bacillus subtilis, Bacillus natto, and lactic bacteria, red yeast rice, brewer's yeast and alcohol yeast, that work to drive one or more fermentation reactions. In addition, one or more sugars may be included in a reaction mixture to facilitate fermentation. The solvent used in forming the reaction mixture can be any suitable solvent, including water and water-based solvents, such as distilled water or purified water and acidic aqueous solvents including, for example, those described herein. In addition, the solvent used in forming a reaction mixture can include one or more non-aqueous solvents, and in one embodiment, the solvent used in a reaction mixture includes up to about 10% non-aqueous solvent, and in an example of such an embodiment the amount of non-aqueous solvent is selected from up to about 1%, up to about 2%, up to about 3%, up to about 5%, up to about 7% and up to about 10% by weight.

A composition described herein, such as a collagen producing composition, an antioxidant composition, and an anti-inflammatory composition, can be isolated from a reaction mixture by any desired extraction, isolation or purification technique. In one embodiment, the composition can be extracted via a supercritical extraction using liquefied carbon dioxide. In another embodiment, the composition can be extracted with an acidic aqueous solution, such as those mentioned herein, and the solvent used in the reaction mixture is removed, such as by drying, to provide an extraction product. In addition to the acidic aqueous solutions already mentioned, aqueous acidic solutions such as formic acid, propionic acid, butyric acid, alpha hydroxyl acid and phosphoric acid solutions, including solutions including a combination of two or more of such acids, may be used to extract or refine a composition isolated from or contained within a reaction mixture. In another embodiment, the compositions described herein can be extracted from the reaction mixture using a vegetable oil or mixture of vegetable oils. For example, vegetable oils that may be used include coconut oil, palm oil, soybean oil, olive oil, rapeseed oil, rice oil, germ oil, corn oil, safflower oil, linseed oil, almond oil, sesame oil, cacao oil, canola oil, grapeseed oil, egoma oil, wheat germ oil, rice bran oil, basil oil, tea oil, primrose oil, pumpkin seed oil, peanut oil, grape oil, hazelnut oil, cottonseed oil, vegetable oils containing extract of persimmon leaves, lycium fruit, chrysanthemum or pine leaves, or other vegetable oils or combinations of vegetable oils suitable for use in foods or cosmetic products. Where a plant extract, such as an extract of persimmon leaves, lycium fruit, chrysanthemum or pine leaves is included in a vegetable oil used for extraction, such an extract may be provided and combined with the vegetable oil using any suitable extraction or formulation techniques. In one embodiment, a plant extract included in a vegetable oil used for extraction of one or more of the compositions as described is derived from a plant grown organically or without agrochemicals.

In one embodiment, a composition as described herein obtained from the reaction mixture may be subjected to one or more subsequent refinement steps. For example, one or more of a composition for promoting collagen production, an antioxidant composition obtained from the reaction mixture, and an anti-inflammatory composition may be subjected to one or more subsequent refinement steps. For instance, a substrate separation procedure may be used to further separate or refine one or more of the compositions. Typically, such a procedure employs a solid phase, such as porous polysaccharides, silicon oxide compounds, polyacrylamides, polystyrenes, polypropylenes and/or styrene-vinyl benzene copolymers. Carriers having particle sizes of 0.1 to 300 micrometers are desirable. The solid phase particles may be surface coated to assist in separation of the desired constituent(s). For example, reverse phase carriers having their surfaces coated with hydrophobic compounds are used for separation of highly hydrophobic substances. Solid phase materials coated with anionic substances are suitable for separation of cationically charged substances, while solid phase materials coated with cationic substances are suitable for separation of anionically charged substances. Even further, if desired, the solid phase materials may be coated with specific antibodies and used as affinity carriers or resins for highly specific separation of targeted substances. Affinity carriers or resins are used for specific preparation of antigens with the use of antigen-antibody reactions. Distributable solid phase materials or resins, such as silica gel (Merck Ltd.), are used for isolation of substances when there are differences in distribution coefficients between the substances and solvents for separation. Known solid phase materials may be used to separate or purify one or more desired xanthone derivatives. For example, known solid phase materials used in a separation or purification process can be reverse phase, adsorptive, or distributable materials, and known solid phase materials can be used as molecular sieves or in ion exchange processes. Specific examples of solid phase materials that may be used in a method for isolating or purifying a composition as described herein include commercially available adsorbtive solid phase materials, such as Diaion (Mitsubishi Chemical Corporation) and XAD-2 or XAD-4 (Rohm and Haas Company), commercially available solid phase materials for molecular sieves, such as Sephadex LH-20 (Amersham Pharmacia), commercially available solid phase materials for distribution, such as silica gel, commercially available ion exchange solid phase materials, such as IRA-410 (Rohm and Haas Company), and commercially available reverse phase materials, such as DM1020T (Fuji Silycia Chemical Ltd.).

Where one or more of the compositions described herein is isolated or further purified using a solid phase as described above, an aqueous solvent, organic solvent or one or more vegetable oils may be added to the extract obtained from the reaction mixture to facilitate the separation process. In one embodiment, the relative amount, as measured by weight, of the solvent may be, for example, between 1 and 40 times the amount of the extracted composition used in the separation procedure. In another such embodiment, the relative amount, as measured by weight, of the solvent may be, for example, between 3 and 20 times the amount of extracted composition used. In another embodiment, the separation procedure may be conducted at a temperature between about 4° C. and about 60° C. In one such embodiment, the separation procedure is conducted at a temperature between about 15° C. and about 50° C.

Where a vegetable oil is used as a solvent in a substrate separation procedure, the vegetable oil may be selected from those described above, and in one such embodiment, a vegetable oil used may be chosen for its medicinal or therapeutic properties. For example, a vegetable oil having pine leaf extract added thereto may be used as a solvent, with the pine leaf extract providing anti-inflammatory and antiseptic action. Where an aqueous solvent is used in a substrate separation procedure, it may be water, such as distilled water, purified water, or acidic acid solutions, including those described herein. Where organic solvents are used in a substrate separation procedure, the solvent may be selected from, for example, one or more of lower alcohols containing water (e.g., hydrous alcohols such as hydrous ethanol), lower alcohols, hexane, ethyl acetate, benzene, triethyl ether, chloroform, esters, ethanol, ketones, such as acetone, and other suitable and commercially available hydrophilic and lipophilic solvents. Examples of suitable alcohols include, but are not limited to, methanol, ethanol, propanol and butanol. Though the various different solvents are described individually, it is to be understood that combinations of the solvents described herein, including combinations of aqueous and organic solvents, may also be employed to achieve separation of a desired constituent or desired process performance characteristics.

Where a substrate separation or purification process is employed, the solid phase as well as the organic solvents or vegetable oils used in the process are preferably suitable for preparation of pharmaceutical or food grade products. Moreover, the solvent or combination of solvents used in any given separation technique will depend on the solid phase material used. For example, when organic solvents are used as solvents for separation, solid phase materials having resistance against such organic solvents are used. In a specific embodiment, an extract obtained from a reaction mixture as described above is subjected to a further separation step wherein Sephadex LH-20 is used as the solid phase and lower alcohols are used as solvents for separation. In another specific embodiment, an extract obtained from a reaction mixture as described above is subjected to a further separation step wherein silica gel is used as the solid phase and chloroform, methanol, acetic acid or liquid mixtures thereof are used as the solvent for separation. In yet a further specific embodiment, an extract obtained from a reaction mixture as described above is subjected to a further separation step, wherein Diaion and DM1020T are used as the solid phase and lower alcohols such as methanol and ethanol or liquid mixtures of lower alcohols and water, are used as the solvent for separation.

Once the extract obtained from the reaction mixture has been subjected to one or more further separation steps, the further separated fraction or fractions providing one or more of the compositions described herein may be subjected to further drying steps to remove the separation solvent used. Such drying steps can be undertaken using known techniques, such as vacuum drying, and equipment that is commercially available. The compositions obtained by such techniques may be in a liquid or powder form.

In one embodiment, one or more compositions described herein may be obtained by providing a reaction mixture that can include a mangosteen grind formed of ground fruit pulp and/or pericarp from the mangosteen fruit, a solvent, such as water, including tap water, distilled water or purified water, a fermentation base, such as soybeans, and one or more biologic constituents, such as one or more microorganisms selected from lactic bacteria, red yeast rice, Bacillus subtilis, including Bacillus natto, brewer's yeast, and alcohol yeast. In one such embodiment, Bacillus natto, such as is commercially available from, for example, Nattomotohonpo, are used in the reaction mixture. Where used, Bacillus natto may be included in the reaction mixture at about 0.001 parts to about 0.03 parts by weight relative to 1 part by weight of the mangosteen grind. Where the amount of Bacillus natto included is less than about 0.001 parts by weight, production of a desired fermentation product may be reduced, while inclusion of more than about 0.03 parts by weight Bacillus natto may initiate a fermentation process that proceeds too rapidly. Where soybeans are used to provide a fermentation base in a method according to the present description, the soybeans may be washed, heated or boiled, and ground. Additionally, in one embodiment, a soybean grind is used as a fermentation base and included in the reaction mixture in an amount of about 3 parts to about 20 parts by weight relative to 1 part by weight of the mangosteen grind. Where the amount of soybean grind is less than 3 parts by weight relative to 1 part by weight of a mangosteen grind, production of a desired fermentation product may be reduced, while inclusion of more than about 20 parts by weight of a soybean grind may also reduce yield of desired fermentation products. In one embodiment, the reaction mixture is fermented at a temperature of about 30° C. to about 48° C. for a period of about 24 hours to about 72 hours. In another such embodiment, the reaction mixture includes 1 part by weight of a mangosteen grind, 20 parts by weight of a soybean grind, and is fermented at a temperature of 39° C. for a period of 70 hours.

In one embodiment, an antioxidant composition as described herein is obtained by providing a reaction mixture that includes a mangosteen grind formed of ground fruit pulp and/or pericarp of the mangosteen fruit, with a solvent, such as water, including tap water, distilled water or purified water, and arginine. The arginine used in such an embodiment may be obtained commercially, and in a specific embodiment is obtained from Ajinomoto Co., Inc. In one embodiment, arginine is added to the reaction mixture at about 0.1 parts to about 2 parts by weight in relation to 1 part by weight of the mangosteen grind. When the amount of arginine added is less than about 0.1 part by weight, production of the desired reaction product(s) may be reduced, while if the amount of arginine added to the reaction mixture is greater than about 1 part by weight, alkalinity of the reaction mixture may increase to a level that results in discoloration of the extracts isolated from the reaction mixture. Once provided, the reaction mixture may be warmed, and in a specific embodiment, the reaction mixture is warmed to a temperature of about 20° C. to about 60° C. In another embodiment, the reaction mixture is warmed to a temperature of about 30° C. to about 50° C. In one embodiment, the reaction mixture is warmed at the temperatures described for a period of about 1 hour to about 24 hours. In one such embodiment, the reaction mixture is warmed for a period of about 3 hours to about 12 hours. After warming and agitation of the reaction mixture, the antioxidant composition produced may be extracted, for example, using an ethanol extraction. The ethanol used in the extraction process may be ethanol suitable for food and pharmaceutical processing, and in one embodiment, the extraction may be carried out at a temperature of about 30° C. to about 90° C. In one such embodiment, the ethanol extraction is carried out at a temperature of about 40° C. to about 80° C.

In another embodiment, an antioxidant composition according to the present description is obtained by providing a reaction mixture that includes a mangosteen grind formed of ground fruit pulp and or pericarp of the mangosteen fruit, peach juice, a lipase, and a solvent, such as water, including tap water, distilled water or purified water. As used herein, “peach juice” refers to juice obtained from a peach fruit, which contains one or more enzymes capable of catalyzing polymerization of polyphenol compounds. In one embodiment, peach juice is added to the reaction mixture at about 0.01 parts to about 1 parts by weight relative to 1 part by weight of the mangosteen grind. When the amount of peach juice added is less than about 0.01 part by weight, production of the desired reaction product(s) may be reduced, while if the amount of peach juice added to the reaction mixture is greater than about 1 part by weight, acidity of the reaction mixture may increase to a level that results in discoloration of the extracts isolated from the reaction mixture. In one embodiment, the lipase included in the reaction mixture is provided at about 0.001 parts to about 0.1 part by weight relative to 1 part by weight of the mangosteen grind. When the amount of a lipase added is less than about 0.001 parts by weight, production of desired reaction product(s) may be reduced, while if the amount of a lipase added is more than about 0.1 part by weight, one or more reactions may proceed at a rate that reduces solubility of reaction products. Once provided, the reaction mixture may be warmed, and in a specific embodiment, the reaction mixture is warmed to a temperature of about 25° C. to about 50° C. In another embodiment, the reaction mixture is warmed to a temperature of about 30° C. to about 40° C. In one embodiment, the reaction mixture is warmed for a period of about 1 hour to about 48 hours. In one such embodiment, the reaction mixture is warmed for a period of about 3 hours to about 20 hours. After warming and agitation of the reaction mixture, the antioxidant composition produced may be extracted, for example, using an glycerin and vegetable oil extraction. In one embodiment, a glycerin extraction may be carried out at a temperature of about 25° C. to about 90° C. In one such embodiment, a glycerin extraction is carried out at a temperature of about 40° C. to about 80° C.

Lipases for use in the methods described herein are also readily obtained through commercial sources. For example, suitable, commercially available lipases include, but are not limited to, Lipozyme and Novozyme 435 manufactured by Novozymes, Lipase PL and Lipase QLM manufactured by Meito Sangyo Co., Ltd., Lipase AY “Amano” 30G, Lipase G “Amano” 50, Lipase F-AF15 and Newlase F3G manufactured by Amano Enzyme Inc.

In any of the methods described herein, the pH of the reaction mixture may be controlled to achieve desired reaction products. For example, in one embodiment, where a lipase is included in the reaction mixture, a pH of about 8 to about 9 may be maintained.

In yet another embodiment, a method for manufacturing an antioxidant composition as described herein may include a fermentation step. In such an embodiment, the reaction mixture may include a mangosteen grind formed of ground fruit pulp and/or pericarp from the mangosteen fruit, a solvent, such as water, including tap water, distilled water or purified water, a fermentation base, such as soybeans, and one or more biologic constituents, such as one or more microorganisms selected from Bacillus natto, lactic bacteria, red yeast rice and Bacillus subtilis, brewer's yeast, and alcohol yeast. In one such embodiment, lactic bacteria, such as those that are commercially available from, for example, Biofermin Pharmaceutical Co., Limited, are used in the reaction mixture. Where used, lactic bacteria may be included in the reaction mixture at about 0.005 parts to about 0.03 parts by weight relative to 1 part by weight of the mangosteen grind. Where the amount of lactic bacteria included is less than about 0.005 parts by weight, production of a desired fermentation product may be reduced, while inclusion of more than about 0.03 parts by weight lactic bacteria may initiate a fermentation process that proceeds too rapidly. Where soybeans are used to provide a fermentation base in a method according to the present description, the soybeans may be washed, heated or boiled and ground. Additionally, in one embodiment, a soybean grind is used as a fermentation base and included in the reaction mixture in an amount of about 3 parts to about 20 parts by weight relative to 1 part by weight of the mangosteen grind. Where the amount of soybean grind is less than 3 parts by weight relative to 1 part by weight of a mangosteen grind, production of a desired fermentation product may be reduced, while inclusion of more than about 20 parts by weight of a soybean grind may also reduce yield of desired fermentation products. In one embodiment, the reaction mixture is fermented at a temperature of about 10° C. to about 40° C. for a period of about 24 hours to 96 hours and at rate of agitation of about 12 rpm to about 120 rpm. In one such embodiment, the reaction mixture is fermented at a temperature of about 20° C. to about 37° C. for a period of about 30 hours to 60 hours.

Where a method for producing an antioxidant composition includes a fermentation step, arginine, such as L-arginine, may be additionally included in the reaction mixture. L-arginine is readily commercially available from sources such as, for example, Ajinomoto Co., Inc. Where included in a reaction mixture for fermentation, arginine may be added at about 0.01 parts to about 0.1 parts by weight relative to 1 part by weight mangosteen grind.

Extraction of an antioxidant composition from a reaction mixture, including reaction mixtures having undergone fermentation, can be carried out using a glycerin extraction process, wherein the glycerin is selected from one or more of the glycerin already described. In one embodiment, the glycerin is added in an amount of about 0.01 to about 3 parts by weight in relation to 1 part by weight of the reaction mixture. Extraction may be achieved by agitation of the oil and reaction mixture at temperatures ranging from about 20° C. to about 50° C. over a period ranging from about 0.5 hours to about to 8 hours. In one embodiment, the glycerin used in the extraction process is a glycerin containing pine leaf extract added in an amount of about 0.02 to about 2 parts by weight in relation to 1 part by weight of a reaction mixture and the glycerin and reaction mixture are agitated at temperatures of about 20° C. to about 50° C. over a period of about 0.5 hours to about to 8 hours. After agitation, supernatant formed by the glycerin phase is collected and residual solvent may be removed. Collection of the phase can be carried out using any standard technique and equipment, and drying or removal of residual solvent, such as water, from the collected glycerin phase can be carried out using standard techniques and equipment. In one embodiment, residual solvent is removed using a commercially available drier, such as the TGD-250LF2 manufactured by TOYOGIKEN Co., Ltd.

In one embodiment, following fermentation of the reaction mixture, a composition for promoting collagen production can be isolated or extracted from the fermented product using a acidic aqueous solution, such as a solution selected from the acidic aqueous solutions described herein. Moreover, extraction of the composition for promoting collagen production from the fermented reaction mixture can be carried out using a combination of acidic aqueous solutions, such as an acetic acid and citric acid solution. In one embodiment, where an acidic aqueous solution is used to extract the composition, the acidic aqueous solution may be added to the fermented reaction mixture at about 0.03 parts to about 10 parts by weight relative to 1 part by weight of the fermented reaction mixture. Following addition of the acidic aqueous solution, the mixture can be agitated at a temperature of about 10° C. to about 50° C. for a period of, for example, about 0.3 hours to about 5 hours. Following agitation, the aqueous phase is separated, collected and dried to provide an extract comprising a composition capable of promoting collagen production. For purposes of drying, a commercially available dryer, such as a TGD-250LF2 manufactured by TOYOGIKEN Co., Ltd., may be used. Alternatively, known spray drying or lyophilization techniques can be used to carry out drying of the extract. The dried composition obtained may be further refined, such as by a chromotography process as described herein.

In one embodiment, extraction of a composition as described herein from a fermented reaction mixture is carried out using an acidic aqueous solution selected from a citric acid solution, an acetic acid solution, and a solution including a combination of citric acid and acetic acid, wherein the acidic aqueous solution is added to the fermented reaction mixture at about 0.03 parts to about 3 parts by weight relative to 1 part by weight of the fermented reaction mixture. In such an embodiment, the mixture may be agitated at temperatures of about 16° C. to about 50° C. for a period of about 0.4 to about 5 hours, followed by separation, collection and drying as described above to obtain a desired collagen promoting composition.

In one embodiment, a method for making an anti-inflammatory composition as described herein includes providing a reaction mixture including palmitic acid, lipase and a mangosteen grind. In such a method, palmitic acid and a lipase are added to a mangosteen grind, followed by warming and extraction with a vegetable oil. In carrying out this method, a mangosteen grind including both mangosteen fruit pulp and pericarp, water, such as tap water, distilled water or purified water, as a solvent, palmitic acid and a lipase are placed in a clean vessel, such as a stainless steel vessel, where they are mixed and warmed. In one embodiment, the palmitic acid is included in the reaction mixture at about 0.02 to about 2 parts by weight in relation to 1 part by weight of the mangosteen grind, and the lipase is included in the reaction mixture at about 0.001 to about 0.1 part by weight in relation to 1 part by weight of the mangosteen grind. The reaction mixture may be warmed, for example, to a temperature of between about 15° C. and about 48° C., and in one such embodiment, the reaction mixture is warmed to a temperature of between about 18° C. and about 39° C. The reaction mixture may be warmed at a desired temperature or within the desired temperature range for between about 2 and about 28 hours, and in one such embodiment, the reaction mixture is warmed for between about 6 and about 24 hours. The reaction mixture may be agitated while it is warmed, and the rate of agitation can be selected from stirring of between about 20 rpm and about 140 rpm. Once the reaction mixture has been agitated and warmed over a desired period of time, agitation can stop and the reaction mixture can be cooled, such as by allowing the mixture to cool to ambient temperature. An anti-inflammatory composition produced by this method can be extracted from the reaction mixture using a vegetable oil or combination of vegetable oils as described above. In one embodiment, the amount of vegetable oil added to carry out an extraction is selected from between about 2 and about 10 parts by weight in relation to 1 part by weight of the mangosteen grind.

The palmitic acid used in a reaction mixture according to the methods described herein may be obtained commercially. For example, palmitic acid extracted from plant oils, such as one or more of coconut oil, rapeseed oil, cotton seed oil, corn oil, safflower oil, sesame oil, rice oil, sunflower oil, or olive oil, or other plant or vegetable oils, is readily available commercially. In a specific example, palmitic acid extracted and refined from plants such as coconut and soybean, available from Ryo Shoku Ltd., The Nisshin Oillio Group, Ltd. and Fuji Oil Co., Ltd. is suitable for use in the methods described herein.

In another embodiment, a method for making an anti-inflammatory composition as described herein includes providing a reaction mixture wherein p-coumaric acid and a lipase are added to a mangosteen grind, followed by warming and extraction with a vegetable oil. In carrying out this method, a mangosteen grind including both mangosteen fruit pulp and pericarp, water, such as tap water, distilled water or purified water, as a solvent, p-coumaric acid and a lipase are placed in a clean vessel, such as a stainless steel vessel, where they are mixed and warmed. In one embodiment, the p-coumaric acid is included in the reaction at about 0.03 to about 3 parts by weight in relation to 1 part by weight of the mangosteen grind, and the lipase is included in the reaction mixture at about 0.001 to about 0.2 parts by weight in relation to 1 part by weight of the mangosteen grind. The reaction mixture may be warmed, for example, to a temperature of between about 14° C. and about 50° C., and in one such embodiment, the reaction mixture is warmed to a temperature of between about 25° C. and about 39° C. The reaction mixture may be warmed at a desired temperature or within the desired temperature range for between about 2 and about 48 hours, and in one such embodiment, the reaction mixture is warmed for between about 6 and about 24 hours. The reaction mixture may be agitated while it is warmed, and the rate of agitation can be selected from stirring of between about 50 rpm and about 140 rpm. Once the reaction mixture has been agitated and warmed over a desired period of time, agitation can stop and the reaction mixture can be cooled, such as by allowing the mixture to cool to ambient temperature. An anti-inflammatory composition produced by this method can be extracted from the reaction mixture using a vegetable oil or combination of vegetable oils as described above. In one embodiment, the amount of vegetable oil added to carry out an extraction is selected from between about 2 and about 10 parts by weight in relation to 1 part by weight of the mangosteen grind.

The p-coumaric acid used in a reaction mixture according to the methods described herein may be extracted from, for example, coffee, green coffee, cocoa, or propolis. As an example, p-coumaric acid manufactured by API Co., Ltd. is suitable for use in the methods described herein.

In another embodiment, a method for making an anti-inflammatory composition as described herein includes providing a reaction mixture wherein sinapic acid and a lipase are added to a mangosteen grind, followed by warming and extraction with a vegetable oil. In carrying out this method, a mangosteen grind including both mangosteen fruit pulp and pericarp, water, such as tap water, distilled water or purified water, as a solvent, sinapic acid and a lipase are placed in a clean vessel, such as a stainless steel vessel, where they are mixed and warmed. In one embodiment, the sinapic acid is included in the reaction mixture at about 0.04 to about 4 parts by weight in relation to 1 part by weight of the mangosteen grind, and the lipase is included in the reaction mixture at about 0.003 to about 0.3 parts by weight in relation to 1 part by weight of the mangosteen grind. The reaction mixture may be warmed, for example, to a temperature of between about 14° C. and about 45° C., and in one such embodiment, the reaction mixture is warmed to a temperature of between about 25° C. and about 37° C. The reaction mixture may be warmed at a desired temperature or within the desired temperature range for between about 2 and about 48 hours, and in one such embodiment, the reaction mixture is warmed for between about 6 and about 24 hours. The reaction mixture may be agitated while it is warmed, and the rate of agitation can be selected from stirring of between about 40 rpm and about 140 rpm. Once the reaction mixture has been agitated and warmed over a desired period of time, agitation can stop and the reaction mixture can be cooled, such as by allowing the mixture to cool to ambient temperature. An anti-inflammatory composition produced by this method can be extracted from the reaction mixture using a vegetable oil or combination of vegetable oils as described above. In one embodiment, the amount of vegetable oil added to carry out the extraction is selected from between about 2 and about 10 parts by weight in relation to 1 part by weight of the mangosteen grind.

The sinapic acid used in methods as described herein may be extracted from, for example, propolis, cinnamon, or clove. As an example, sinapic acid manufactured by Anri Japan is suitable for use in the methods described herein.

In another embodiment, a method for making an anti-inflammatory composition as described herein includes providing a reaction mixture wherein xanthone-carboxylic acid and a lipase are added to a mangosteen grind, followed by warming and extraction with a vegetable oil. In carrying out this method, a mangosteen grind including both mangosteen fruit pulp and pericarp, water, such as tap water, distilled water or purified water, as a solvent, xanthone-carboxylic acid, and a lipase are placed in a clean vessel, such as a stainless steel vessel, where they are mixed and warmed. In one embodiment, the xanthone-carboxylic acid is included in the reaction mixture at about 0.03 to about 5 parts by weight in relation to 1 part by weight of the mangosteen grind, and the lipase is included in the reaction mixture at about 0.001 to about 0.3 part by weight in relation to 1 part by weight of the mangosteen grind. The reaction mixture may be warmed, for example, to a temperature of between about 14° C. and about 60° C., and in one such embodiment, the reaction mixture is warmed to a temperature of between about 25° C. and about 45° C. The reaction mixture may be warmed at a desired temperature or within the desired temperature range for between about 2 and about 48 hours, and in one such embodiment, the reaction mixture is warmed for between about 6 and about 24 hours. The reaction mixture may be agitated while it is warmed, and the rate of agitation can be selected from stirring of between about 40 rpm and about 140 rpm. Once the reaction mixture has been agitated and warmed over a desired period of time, agitation can stop and the reaction mixture can be cooled, such as by allowing the mixture to cool to ambient temperature. An anti-inflammatory composition produced by this method can be extracted from the reaction mixture using a vegetable oil or combination of vegetable oils as described above. In one embodiment, the amount of vegetable oil added to carry out an extraction is selected from between about 2 and about 10 parts by weight in relation to 1 part by weight of the mangosteen grind.

The xanthone-carboxylic acid used in methods as described herein may be extracted from, for example, Hypericum perforatum. As an example, xanthone-carboxylic acid manufactured by Anri Japan is suitable for use in the methods described herein.

In yet another embodiment, a method for manufacturing an anti-inflammatory composition as described herein may include a fermentation step. In such an embodiment a microorganism, such as Bacillus natto, is added to a mangosteen grind for fermentation. An anti-inflammatory composition produced as part of the fermentation process can be extracted from the fermented product with vegetable oil. In an embodiment of a production method that includes a fermentation step, ground soybean is added to the mangosteen material used in the reaction mixture. The soybeans used in such a method can be sourced from any suitable commercial source, and in one example, the Bacillus natto used in the methods described herein may be manufactured by Nattomotohonpo. As described herein, Bacillus natto is a species of Bacillus subtilis that is used for processing of natto and other foods. In one embodiment, the amount of soybean added to the reaction mixture ranges from about 0.6 to about 5 parts by weight, and the amount of Bacillus natto added to the reaction mixture ranges from about 0.001 to about 0.03 part by weight, both relative to 1 part by weight of the mangosteen grind used in the reaction mixture. The fermentation is carried out using standard equipment and water, such as tap water, distilled water or purified water, may be used as a solvent in the reaction mixture. Where the method includes a fermentation step, the fermentation may be carried out by warming the reaction mixture to a temperature of between about 30° C. and about 50° C. over a period of time ranging from about 24 hours to about 72 hours. Once the fermentation step has been completed, the reaction mixture may be diluted with water, for example, tap water, distilled water, or purified water, which can facilitate extraction of an anti-inflammatory composition.

Extraction of an anti-inflammatory composition can be carried out using a vegetable oil extraction process, wherein the vegetable oil is selected from one or more of the vegetable oils already described. In one embodiment, the vegetable oil is added in an amount ranging from about 0.03 to about 3 parts by weight in relation to 1 part by weight of the fermented reaction mixture. Extraction may be achieved by agitation of the oil and fermented reaction mixture at temperatures ranging from about 20° C. to about 50° C. over a period ranging from about 1 hour to about to 6 hours. In one embodiment, the vegetable oil used in the extraction process is a vegetable oil containing pine leaf extract. After agitation, supernatant formed by the vegetable oil phase is collected to remove water. Collection of the oil phase can be done using any standard technique and equipment, and drying or removal of water from the collected oil phase can be carried out using, for example, a commercially available drier, such as the TGD-250LF2 manufactured by TOYOGIKEN Co., Ltd.

### Methods of Using Compositions for Promoting Collagen Production

The compositions described herein are useful in a wide variety of applications, such as, for example, in pharmaceuticals, food preparations, cosmetic products, hygienic devices, clothing, fibers, plastics processing, and environmental protective materials.

When used in pharmaceuticals, the compositions according to the present description are suitable for veterinary and human use and may be dosed systemically or topically as an anti-allergenic agent, to promote collagen production or to inhibit, treat or alleviate diseases, conditions or disorders characterized by reduced collagen production. The compositions provided herein may additionally exhibit anti-inflammatory properties and antimutagenic properties and may be delivered to a subject in order to inhibit, treat or alleviate diseases, conditions or disorders associated with or accompanied by swelling, mutagenasis, irritation, or inflammation, including diseases, conditions or disorders associated production of inflammatory cytokines, systemic inflammation, such as fever, rubor or edema, and localized inflammation such as pollinosis, rhinitis, sinusitis, pneumonia, encephalitis, pharyngitis, bronchitis, keratitis, enteritis, gastritis, duodenitis, colitis, hepatitis, nephritis, urocystitis, pancreatitis, neuritis, myositis, arteritis, dermatitis, atopic dermatitis and inflammation or dermatitis due to sunburn.

When used in food preparations, the compositions described herein are suitable for veterinary and human use and may be used as a dietary or food supplement for promoting collagen production, alleviating dermatitis, muscle pain or fatigue, and to facilitate maintenance of liver function.

The compositions described herein provide hemangiecstasis and lymphangiecstasis to the skin. When used in personal care products, such as, for example, cosmetic products, the compositions described herein are suitable for veterinary and human use and may be incorporated into a cosmetic product, quasi-drug or aesthetic oil for promoting collagen production, and slowing the formation of, reducing the appearance of, reducing or alleviating wrinkles, spots, dullness or sagging of the skin. The compositions described herein may also be used in a cosmetic product, quasi-drug or aesthetic oil for reducing the appearance of or preventing cellulite and wrinkles formed irregularly on the skin surface, and alleviating symptoms associated with sunburn, inflammation, atopic dermatitis, or production of inflammatory cytokines.

In yet further applications, the compositions described herein may be incorporated into fibers used for clothing or materials, such as plastic materials, used in the production of various consumer products. Fibers to which one or more of the xanthone derivatives described herein have been added may be used in clothing. In one embodiment, such clothing may be any article of clothing that makes direct contact with the wearer's skin, such as undergarments, swim wear, socks, or pajamas. Alternatively, compositions as described herein may also be incorporated into other fabrics that come into direct contact with a user's skin, such as furniture coverings, sheets, bath towels, etc. Incorporation of the compositions described herein into fibers and materials that come in contact with human or animal users facilitates production of consumer products that may themselves work to promote collagen production as they come in contact with the user.

### Formulations Including Composition for Promoting Collagen Production

Formulations including collagen promoting compositions as described herein are also provided. In one embodiment, the formulation is a food product including a composition as described herein and a juice extract, wherein the juice extract is included at about 0.001 parts to about 1 part by weight relative to 1 part by weight of the composition capable of promoting collagen production. The juice extract may be obtained from, for example, the pulps, pericarps, seeds, etc. of fruits such as apple, pear, cranberry, blueberry, strawberry, raspberry, cherry, tangerine, orange, fig, apricot, mango, melon, grape, loquat, banana, watermelon, durian and Cucurbitaceae, which may be organically cultivated or cultured. The juice extract may be in liquid, freeze-dried or powdered form and made from a grind of such fruits. Additionally, in order to obtain a food product as described herein, the composition for promoting collagen production and the juice extract may be mixed and then warmed. In one such embodiment, temperatures for warming are selected from between about 25° C. and about 48° C., and times for warming are selected from between about 3 hours and about 48 hours. When the temperatures for warming are below 25° C., sufficient mixing of the constituents may not occur, while warming temperatures above 48° C. may result in browning of the formulation due to, for example, oxidation. Similarly, warming times of less than 3 hours may not be sufficient to obtain a suitable mixture, while warming times that exceed 48 hours may result in browning of the formulation due to, for example, oxidation. A food composition as described herein may be prepared in the form of a powder, tablet, liquid (drinkables, and the like), capsule, etc. Also, additional well known materials, such as food grade bases, excipients, additives, auxiliary materials, and preservatives, may be included in the food product as desired to achieve a product such as a cake, cracker, cookie, etc., of a particular composition, taste, shelf life, etc.

A food product according to the present description may be formulated, for example, as a dietary supplement, and can be ingested several times a day. In one embodiment, the food product is ingested in an amount of between about 0.2 g and about 10 g daily. In another embodiment, the food product is ingested in an amount of between about 0.3 g and about 6 g daily. In yet another embodiment, the food product is ingested in an amount of between about 0.5 g and about 4 g daily.

In another embodiment, a formulation of a composition as described herein may be a personal care product, such as a cosmetic product, including for example, a skin refresher, cream, ointment, lotion, emulsion pack, oil, soap, face wash, perfume, cologne, bath agent, shampoo, conditioner, etc. The cosmetic product may be in any form and used as a solution, cream, paste, gel, suspension, solid or powder. Further, a cosmetic product formulated using the compositions described herein may be used on humans for skin care products, skin-lightening products, hair-cleaning agents, skin treatment agents, hair dyes, hair growth formulas, baldness remedies, and body washes. The cosmetic products described herein may also be used in the veterinary context.

A personal care product according to the present description includes a composition for promoting collagen production as described herein. A personal care product according to the present description may be applied to the skin up to several times a day. In one embodiment, the personal care product is applied in a total daily amount of about 0.01 g to about 10 g. In another embodiment, the personal care product is applied in a total daily amount of about 0.05 g to about 3 g. In yet a further embodiment the personal care product is applied in a total daily amount of about 0.1 g to about 2 g. In particular applications, a personal care product as described herein may be applied to assist in improving sunburn, alleviate pollinosis, alleviate dermatitis, alleviate muscle pain or fatigue, improve wrinkles, dullness, or spots of the skin, reduce the appearance of cellulite, facilitate wound healing, reduce inflammation due to sunburn or skin irritants, or to reduce atopic dermatitis or chemical hypersensitivity due to chemicals and allergens.

In one embodiment, a personal care product includes a juice extract. Where included, a juice extract may be included at about 0.02 parts to about 2 parts by weight in relation to 1 part by weight of the composition for promoting collagen production. Additionally, where included, the juice extract described herein may be obtained from, for example, the pulps, pericarps, seeds, etc., of fruits such as apple, pear, cranberry, blueberry, strawberry, raspberry, cherry, tangerine, orange, fig, apricot, mango, melon, grape, loquat, banana, watermelon, durian and Cucurbitaceae, which may be are organically cultivated or cultured. In one embodiment, the juice extract may be in liquid, freeze-dried or powdered form and made from a grind of such fruits.

In formulating a personal care product, such as a cosmetic product, as described herein, the combined constituents included in the formulation may be warmed after mixing. In one such embodiment, temperatures for warming are selected from between about 25° C. and about 49° C., and times for warming are selected from between about 3 hours and about 60 hours. When the temperatures for warming are below 25° C., sufficient mixing may not occur, while warming temperatures above 49° C. may result in browning of the formulation due to, for example, oxidation. Similarly, warming times of less than 3 hours may not be sufficient to obtain a desired mixture, while warming times that exceed 60 hours may result in browning of the formulation due to, for example, oxidation.

The personal care formulations described herein may include additional materials, as desired. For example, a cosmetic composition according to the present description may include one or more additional oils, surface active agents, vitamins, UV absorbers, thickeners, humectants, auxiliary materials, etc., as are well known in the art, to provide a formulation exhibiting desired characteristics.

In a further embodiment, a pharmaceutical formulation including a composition for promoting collagen production as described herein is provided. A pharmaceutical formulation as contemplated herein may be prepared for oral, topical, or parenteral delivery and may include a xanthone derivative as described herein in combination with one or more binders, excipients, swelling agents, lubricants, preservatives, pigments, sweeteners, flavors, etc. that provide a pharmaceutical composition of desired characteristics. The pharmaceutical formulation may be prepared as a tablet, capsule, syrup, ointment, cream, emulsion, suspension, lyophilized powder, etc. Where the pharmaceutical formulation is prepared for topical administration, a base such as Vaseline®, paraffins, fats and oils, lanolins and macrogels, may be used. Where the pharmaceutical formulation is prepared as a lyophilized powder, the composition may be reconstituted using any suitable vehicle, such as sterile water, distilled water, or physiological saline.

### Methods of Using Antioxidant Compositions

The antioxidant compositions described herein are useful in a wide variety of applications, including as pharmaceuticals, food preparations, personal care products, including cosmetic products, hygienic devices, clothing, fibers, plastics processing, and environmental protective materials.

For pharmaceutical products, the antioxidant compositions described herein may be dosed systemically or topically as an anti-inflammatory agent, an antimutagenic agent, a lipolytic agent, an anticellulite agent, an agent for reducing or preventing wrinkles, spots or dullness of the skin, an anti-hyperlipidemic agent, an anti-arteriosclerotic agent, or an agent for prevention or improvement of metabolic syndromes, cancers, adult diseases, etc. In the veterinary context, the antioxidant compositions described herein may be used as an anti-inflammatory agent, a lipolytic agent, an antiobesity agent, an agent for reducing or preventing wrinkles, spots or dullness of the skin, a lipotropic agent, an anti-hyperlipidemic agent, an antiarteriosclerotic agent, an anticancer agent, etc.

For food products, the antioxidant compositions described herein may be used as a dietary or food supplement for preventing or suppressing development of cancers, improving wrinkles, spots or dullness of the skin, improving or reducing the appearance of cellulite, reducing local fat, or ameliorating metabolic syndromes and adult diseases, etc. Also, where used as a supplement in the veterinary context, the antioxidant compositions described herein may be provided to pets to improve skin health, reduce the appearance of wrinkles, spots or dullness of the skin, or as a supplement for controlling fatty livers, improving hyperlipidemia, controlling arteriosclerosis, preventing cancers, etc.

For personal care products, such as cosmetic products, the antioxidant compositions may be incorporated into a cosmetic product, quasi-drug or aesthetic oil for improving and preventing wrinkles, spots, dullness or sagging of the skin. The antioxidant compositions described herein may also be used in a cosmetic product, quasi-drug or aesthetic oil for removing or preventing cellulite and wrinkles formed irregularly on the skin surface due to oxidation. The antioxidant compositions described herein may also be incorporated into a veterinary cosmetic product used for animals.

In yet further applications, the antioxidant compositions described herein may be incorporated into fibers used for clothing or materials, such as plastic materials, used in the production of various consumer products. Fibers to which one or more of the antioxidant compositions described herein have been added may be used in clothing. In one embodiment, such clothing may be any article of clothing that makes direct contact with the wearer's skin, such as undergarments, swim wear, socks, or pajamas. Alternatively, xanthone derivatives as described herein may also be incorporated into other fabrics that come into direct contact with a person's skin, such as furniture coverings, sheets, bath towels, etc. Incorporation of an antioxidant composition as described herein into fibers and materials that come in contact with human or animal users facilitates production of consumer products that may themselves work to alleviate conditions associated with oxidative action in or on the skin.

### Formulations Including Antioxidant Compositions

Formulations for administration, consumption or other delivery of the antioxidant compositions described herein are also provided. In one embodiment, the formulation is a food product including a antioxidant composition as described herein and a procyanidin at about 0.001 parts to about 0.3 parts by weight in relation to 1 part by weight of the antioxidant composition.

In another embodiment, a formulation as described herein may be a personal care product, such as cosmetic product, including, for example, a skin refresher, cream, ointment, lotion, emulsion pack, oil, soap, face wash, perfume, cologne, bath agent, shampoo, conditioner, etc. The personal care product may be in any form and used as a solution, cream, paste, gel, suspension, solid or powder. Further, the personal care products described herein may be used on humans in skin care products, skin-lightening products, hair-cleaning agents, skin treatment agents, hair dyes, hair growth formulas, baldness remedies, and body washes. The personal care products described herein may also be used in the veterinary context.

The personal care products described herein may be applied to the skin up to several times a day. In one embodiment, a cosmetic product is applied in a total daily amount of about 0.01 g to about 10 g. In another embodiment, a cosmetic product is applied in a total daily amount of about 0.05 g to about 3 g. In yet a further embodiment, a cosmetic product is applied in a total daily amount of about 0.1 g to about 2 g. Application of the personal care products described herein works to provide an antioxidant effect. In particular applications, a personal care product as described herein may be applied to assist in improving sunburn, to reduce the formation, size or appearance of wrinkles, dullness, or spots of the skin, to reduce the formation or appearance of cellulite, to facilitate wound healing, to reduce oxidation due to sunburn, or to reduce atopic dermatitis or chemical hypersensitivity due to chemicals and allergens.

In one embodiment, a personal care product includes an antioxidant composition as provided herein and a procyanidin in an amount of about 0.05 parts to about 0.5 parts by weight relative to 1 part by weight of the antioxidant composition. In one such embodiment, the personal care product additionally includes a juice extract. Where included, a juice extract may be included at about 0.05 parts to about 1 part by weight in relation to 1 part by weight of the antioxidant composition. Additionally, where included, the juice extract described herein may be obtained from, for example, from the pulps, pericarps, seeds, etc. of fruits such as apple, pear, cranberry, raspberry, cherry, tangerine, orange, fig, apricot, mango, melon, grape, loquat, banana, watermelon and Cucurbitaceae, which may be organically cultivated or cultured. In one embodiment, the juice extract may be in liquid, freeze-dried or powdered form and made from a grind of such fruits.

In formulating a personal care product as described herein, the formulation may be warmed after mixing. In one such embodiment, temperatures for warming are selected from between about 25° C. and about 55° C., and times for warming are selected from between about 6 hours and about 48 hours. When the temperatures for warming are below 25° C., sufficient mixing may not occur, while warming temperatures above 55° C. may result in browning of the formulation due to, for example, oxidation. Similarly, warming times of less than 6 hours may not be sufficient to obtain desired mixed products, while warming times that exceed 48 hours may result in browning of the formulation due to, for example, oxidation.

The personal care products described herein may include additional materials, as desired. For example, a cosmetic composition according to the present description may include one or more additional oils, surface active agents, vitamins, UV absorbers, thickeners, humectants, auxiliary materials, etc., as are well known in the art, to provide a composition exhibiting desired characteristics.

### Methods of Using Anti-inflammatory Compositions

The anti-inflammatory compositions described herein are useful in a wide variety of applications, including as pharmaceuticals, food preparations, personal care products, including cosmetic products, hygienic devices, clothing, fibers, plastics processing, and environmental protective materials.

In the pharmaceutical context, the anti-inflammatory compositions described herein may be dosed systemically or topically as an anti-inflammatory agent, a lipolytic agent, an anticellulite agent, an agent for reducing or preventing wrinkles, a lipotropic agent, an anti-hyperlipidemic agent, an anti-arteriosclerotic agent, or an agent for prevention or improvement of metabolic syndromes, cancers, conditions associated with reduced collagen production, adult diseases, etc. In the veterinary context, the anti-inflammatory compositions described herein may be used as a lipolytic agent, an antiobesity agent, an agent for reducing or preventing wrinkles, a lipotropic agent, an anti-hyperlipidemic agent, an antiarteriosclerotic agent, an anticancer agent, etc.

In food preparations, the anti-inflammatory compositions described herein may be used as a dietary or food supplement for improving wrinkles, improving or reducing the appearance of cellulite, reducing local fat, or ameliorating metabolic syndromes, improving conditions associated with reduced collagen production adult diseases, etc. Also, where used as a supplement in the veterinary context, the anti-inflammatory compositions described herein may be provided to pets to improve skin health, reduce the appearance of wrinkles, or as a supplement for controlling fatty livers, improving hyperlipidemia, controlling arteriosclerosis, improving collagen production, preventing cancers, etc.

For cosmetic products, the anti-inflammatory compositions described herein may be incorporated into a cosmetic product, quasi-drug or aesthetic oil for improving and reducing, reducing the appearance of, or inhibiting the formation of wrinkles or skin sagging resulting from inflammation or associated with a reduction in collagen production. The anti-inflammatory compositions described herein may also be used in a cosmetic product, quasi-drug or aesthetic oil for removing or preventing cellulite and wrinkles formed irregularly on the surface due to inflammation or to reduced collagen production. The anti-inflammatory compositions described herein may also be incorporated into a veterinary cosmetic product used for animals.

In yet further applications, the anti-inflammatory compositions described herein may be incorporated into fibers used for clothing or materials, such as plastic materials, used in the production of various consumer products. Fibers to which one or more of the anti-inflammatory compositions described herein have been added may be used in clothing. In one embodiment, such clothing may be any article of clothing that makes direct contact with the wearer's skin, such as undergarments, swim wear, socks, or pajamas. Alternatively, anti-inflammatory compositions as described herein may also be incorporated into other fabrics that come into direct contact with a person's skin, such as furniture coverings, sheets, bath towels, etc. Incorporation of the anti-inflammatory compositions described herein into fibers and materials that come in contact with human or animal users facilitates production of consumer products that may themselves work to alleviate conditions associated with inflammation or a reduction in collagen production.

### Formulations Containing Anti-inflammatory Compositions

Formulated products for administration or delivery of the anti-inflammatory compositions described herein are also disclosed herein. In one embodiment, the formulation is a personal care product, such as a cosmetic product. A cosmetic product as formulated herein may be in the form of a skin refresher, cream, ointment, lotion, emulsion pack, oil, soap, face wash, perfume, cologne, bath agent, shampoo, conditioner, etc. The cosmetic product may be in any form and used as a solution, cream, paste, gel, solid or powder. Further, the cosmetic products described herein may be used on humans in skin care products, skin-lightening products, hair-cleaning agents, skin treatment agents, hair dyes, hair growth formulas, baldness remedies, and body washes. The cosmetic products described herein may also be used in the veterinary context.

The cosmetic products described herein may be applied to the skin up to several times a day. In one embodiment, the cosmetic product is applied in a total daily amount of about 0.01 g to about 10 g. In another embodiment, the cosmetic formulation is applied in a total daily amount of about 0.05 g to about 3 g. In yet a further embodiment, the cosmetic formulation is applied in a total daily amount of about 0.1 g to about 2 g. Application of the cosmetic products described herein works to provide an anti-inflammatory effect and improve collagen production. In particular applications, the cosmetic composition described herein may be applied to assist in wound healing or to reduce atopic dermatitis or chemical hypersensitivity due to chemicals and allergens.

In one embodiment, a cosmetic formulation described herein contains about 0.05 parts to about 0.8 parts by weight of a first vegetable oil, as described herein, wherein the vegetable oil contains chrysanthemum blossom extract, and about 0.05 parts to about 0.8 parts by weight of a second vegetable oil, as described herein, wherein the second vegetable oil contains one or more extract selected from pine leaf extract, extract of persimmon leaf, chrysanthemum blossom extract, and lycium fruit extract, with the relative weights of both the first vegetable oil and the second vegetable oil being measured in relation to 1 part by weight of an anti-inflammatory composition as described herein. In an alternative embodiment, the cosmetic product comprises a formulation containing about 0.05 parts to about 0.8 parts by weight of a vegetable oil, as described herein, wherein the vegetable oil contains one or more extract selected from pine leaf extract, extract of persimmon leaf, chrysanthemum blossom extract, and lycium fruit extract, with the relative weight of the vegetable oil being measured in relation to 1 part by weight of an anti-inflammatory composition as described herein. Such cosmetic products may be applied topically as an anti-inflammatory formulation or a composition useful in improving skin wrinkles or sagging.

The vegetable oil containing chrysanthemum blossom extract used in a cosmetic formulation as described herein may be an oil that is extracted by adding a vegetable oil to a chrysanthemum blossom grind. Examples of vegetable oils suitable for use in a cosmetic composition as described herein include oils suitable for food or cosmetic products such as coconut oil, palm oil, soybean oil, olive oil, rapeseed oil, rice oil, germ oil, corn oil, safflower oil, linseed oil, almond oil, sesame oil, cacao oil, canola oil, grapeseed oil, egoma oil, wheat germ oil, rice bran oil, basil oil, tea oil, primrose oil, pumpkin seed oil, peanut oil, grape oil, hazelnut oil and cottonseed oil. Vegetable oils containing chrysanthemum blossom extract are manufactured by and available from, for example, Toyo Hakko Co., Ltd. Vegetable oils containing pine leaf extract are obtained by grinding pine leaves by any suitable process, such as by using a commercially available grinder, followed by extraction with a vegetable oil. Treating the ground pine leaves as a raw material with cellulases, such as ONOZUKA R-10 and Y-NC manufactured by Yakult Pharmaceutical Industry Co., Ltd., Cellulase A “Amano” 3 and Cellulase T “Amano” 4 manufactured by Amano Enzyme Inc., may be carried out to increase extraction efficiency.

In formulating a cosmetic product as described herein, the formulation may be warmed after mixing. In one such embodiment, temperatures for warming are selected from between about 30° C. and about 45° C., and times for warming are selected from between about 6 hours and about 40 hours. When the temperatures for warming are below 30° C., sufficient mixing may not occur, while warming temperatures above 45° C. may result in browning of the formulation due to, for example, oxidation. Similarly, warming times of less than 6 hours may not be sufficient to obtain desired mixing, while warming times that exceed 40 hours may result in browning of the formulation due to, for example, oxidation.

The cosmetic formulations described herein may contain additional materials, as desired. For example, a cosmetic product according to the present description may include one or more additional oils, surface active agents, vitamins, UV absorbers, thickeners, humectants, auxiliary materials, etc., as are well known in the art, to provide a composition exhibiting desired characteristics.

### EXAMPLES

The embodiments as described above will now be described by way of examples and experiments. The description below is only intended to be illustrative and practices can be carried out in modified forms.

### Example 1

### Collagen Promoting Composition Obtained Through Fermentation

Pericarps of mangosteen fruit cultivated in Thailand were sun-dried, and 1.5 kg of the dried mangosteen pericarps were ground by a DM-6 grinder manufactured by Nakayamalabo Corporation. The grind was placed in a clean culture tank, and 3.1 L of distilled water were added to create a reaction mixture. Soybeans produced in China were washed, warmed at 37° C. for 30 minutes, and ground, and 2.1 kg of the soybean grind was added to the reaction mixture. 21 g of Bacillus natto manufactured by Nattomotohonpo were added to the reaction mixture, and fermentation of the reaction mixture was carried out with agitation at 37° C. for 42 hours.

3.7 L of distilled water were added to the tank after fermentation to provide a fermented product. 5 L of an aqueous solution containing 0.1% acetic acid and 0.5% citric acid were added to the fermented product and it was agitated at 30° C. for 2 hours. The mixture was left still to allow separation and collection of an aqueous layer and, after filtering, was placed in a dryer (TGD-250LF2, TOYOGIKEN Co., Ltd.) to obtain the desired composition as a powder. This powder was designated the composition of Example 1.

### Example 2

### Characterization of Composition of Example 1

The composition of Example 1 was characterized by HPLC and HPLC-MS. HPLC experiments were performed using a Waters Alliance System equipped with a vacuum degasser, quaternary solvent mixing, autosampler, and a Waters 996 diode array detector. UV spectra were collected across the range of 200-400 nm, extracting 254 nm for chromatograms. Waters Millennium32 software was utilized for instrument control, data collection, and data processing. The column was a Waters Nova-Pak C-8 (3.9×150 mm). The mobile phases consisted of A: 0.1% formic acid in water and B: methanol; optimal separations were achieved using a gradient of 65 to 90% B over 0-30 min at the flow rate of 1.0 mL/min. Injection volume for all the samples and standards was 10 μL. (Published reference: Edward B Walker, 2007, “HPLC analysis of selected xanthones in mangosteen fruit,” J. Sep. Sci. 30, 1229-1234.) HPLC-MS experiments were conducted on a Hewlett-Packard HPLC-MS using ESI positive mode with the same column, gradient, and flow rates.

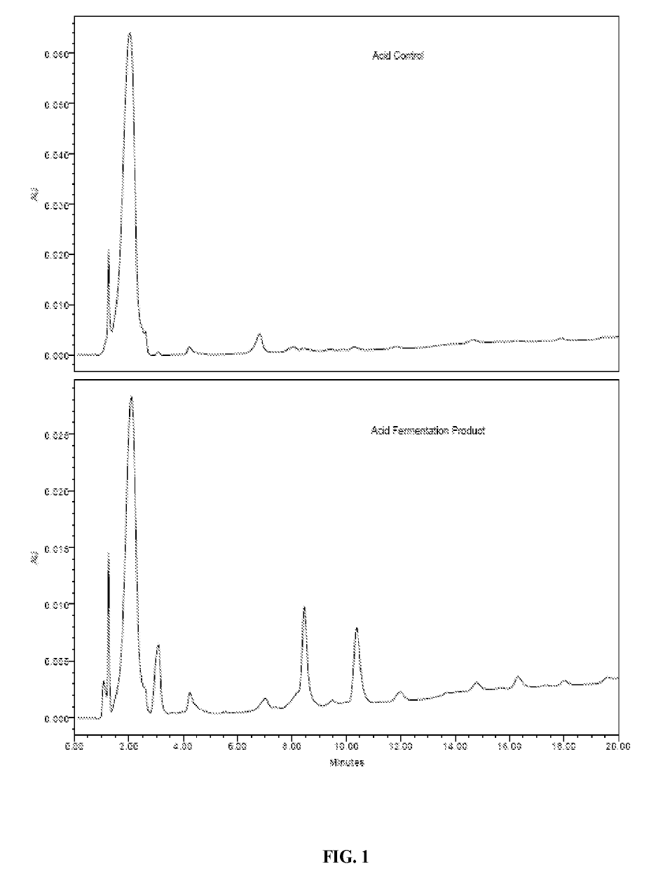

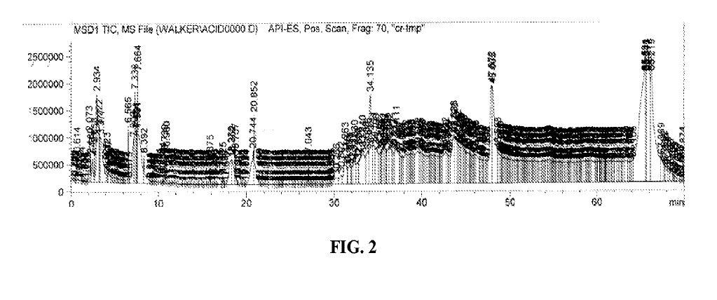

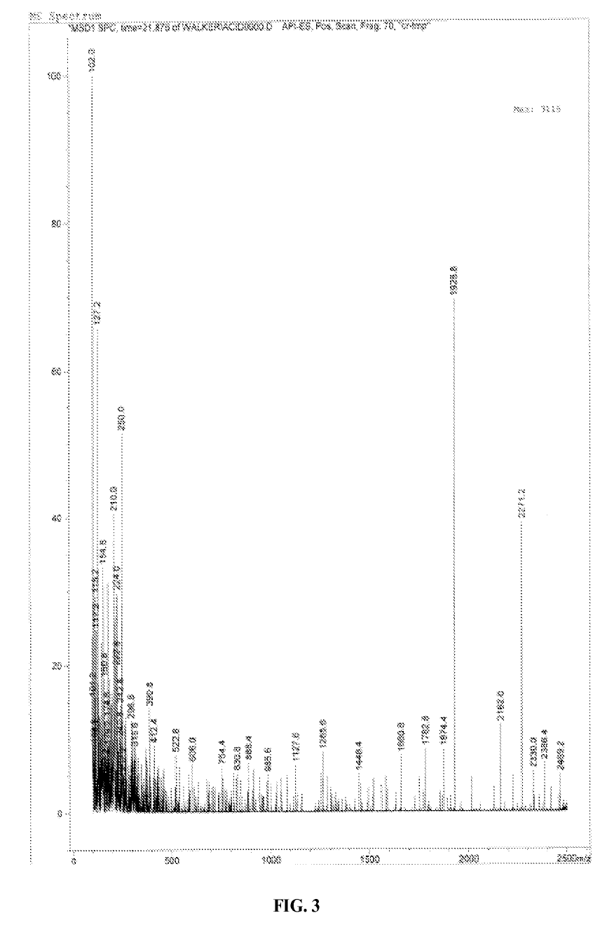

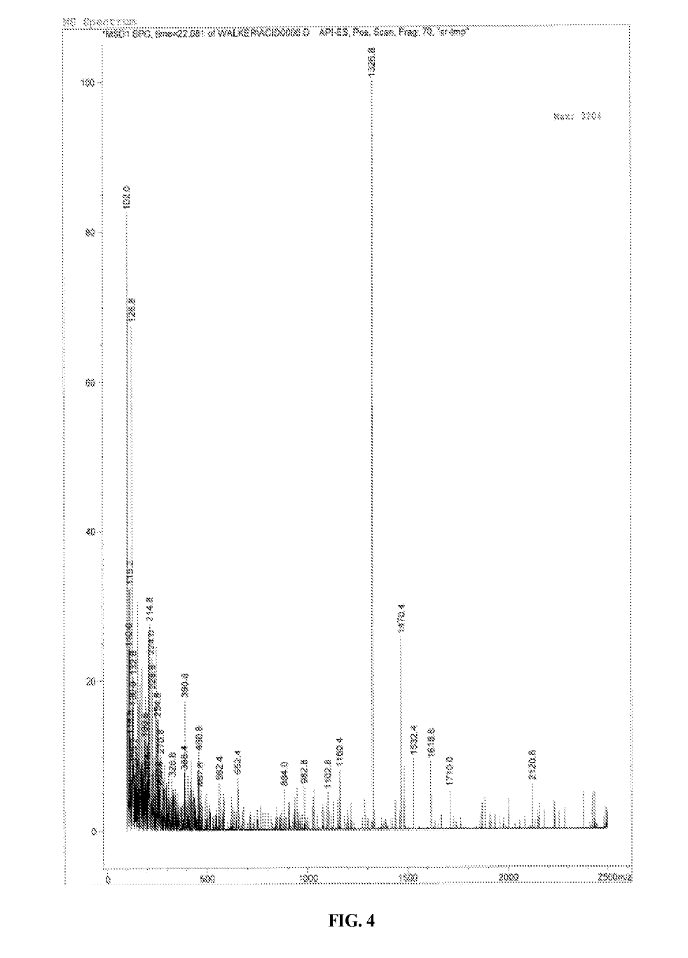

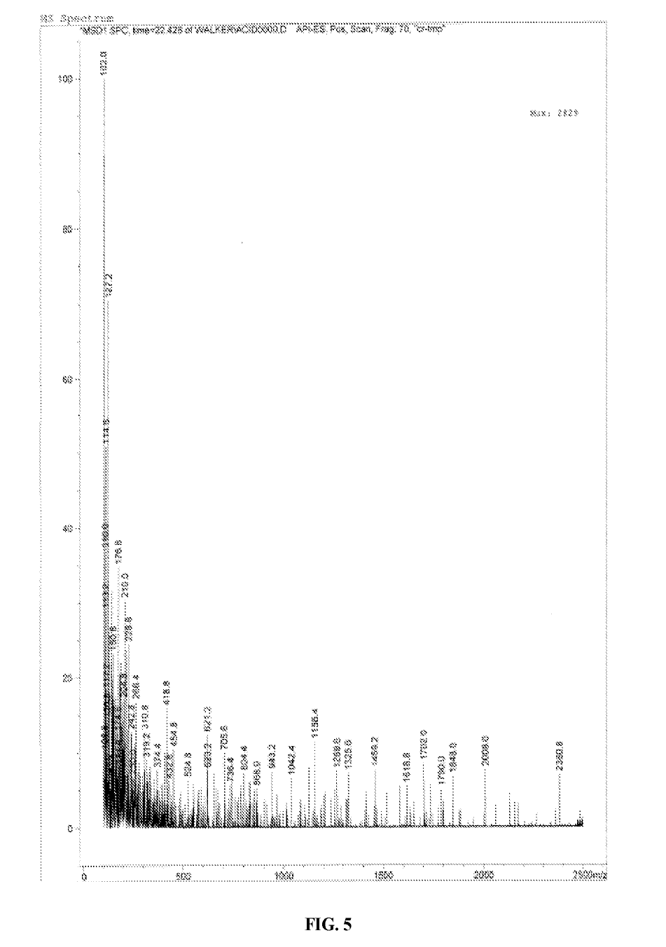

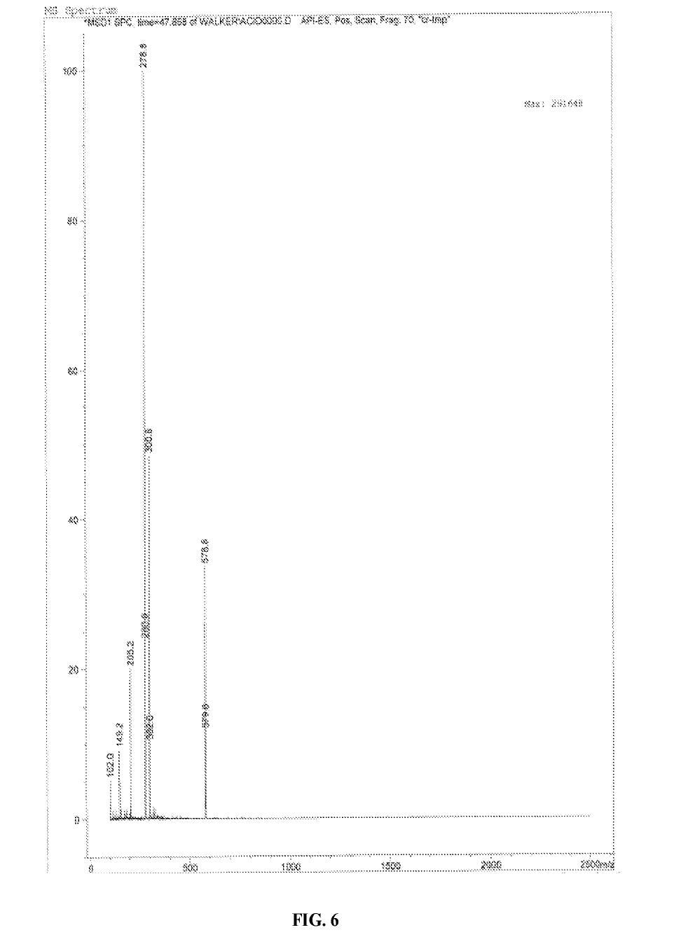

The composition of Example 1 was only slightly soluble in methanol and forms a soft, gelatin precipitate when mixed with methanol (1:1). HPLC chromatograms of the composition of Example 1 showed two peaks at retention times of approximately 8.5 and 10.4 minutes that were not present in a control sample (See chromatogram diagrams in FIG. 1). HPLC-MS chromatograms revealed even more different peaks in the composition of Example 1 that were not observed with ultra-violet detection at 254 nm. Specifically, chromatographic peaks observed in “total ion chromatograph” mode showed significant peaks in the composition at 21.876, 22.081, 22.428, and 47.868 minutes, as shown in FIG. 2. The individual mass spectrum of each of these peaks is also shown in FIGS. 3, 4, 5, and 6 respectively.

### Example 3

### Evaluation of Human Skin Fibroblasts Proliferation and Collagen Production

Human skin derived fibroblasts were purchased from Toyobo Co., Ltd. (Japan). The cells were cultivated with cultivation medium and growth. An aliquot of cells, 40,000 cells, were inoculated into the plastic culture dish, and incubated for 24 hours at 37° C. in an atmosphere of 5% carbon dioxide. The composition of Example 1 was dissolved in sterilized saline solution at a concentration of 0.1 mg/ml. This solution formed using the composition of Example 1 sample was added to the cell culture and cultivation of the fibroblasts was continued. 24 hours after addition of the solution formed using the composition of Example 1, cells from the cell culture were isolated by trypsin and EDTA treatment, the number of live cells were then counted under a microscope after staining with trypan blue dye. The cells were then sonicated and homogenized in a phosphate-buffered saline solution, and the type I collagen content in the homogenate was determined by ELISA kit (Chondrex Co. Ltd.). The live cell count obtained after culturing of the cells in the presence of the solution formed using the composition of Example 1 was 116% of the live cell count obtained in a cell culture receiving only a saline solution as a control. In addition, in the cell culture grown in the presence of the solution formed using the composition of Example 1, production of type I collagen was 300% of the type I collagen production obtained in a cell culture receiving only a saline solution as a control.

### Example 4

### Production of a Cosmetic Product

100 g of the composition obtained in Example 1 were placed in a mixer for cosmetic processing (Bohle container mixer, Kotobuki Industries Co., Ltd.), and 10 g of grape juice extract manufactured by Kikkoman Corporation were added. The mixture was warmed at 38° C. for 33 hours with agitation and then cooled to obtain a composition. The composition was placed in the mixer and 700 g of beeswax (API Co., Ltd.), and 1 g of squalene (Nippon Suisan Kaisha, Ltd.) was added and mixed to obtain a cosmetic preparation as a cream. The cream was designated the cream of Example 4.

### Example 5

### Anti-Wrinkle Clinical Study Using the Cream of Example 4

Using the cream obtained in Example 4, 10 women, ages ranging from 24 to 60, were subjected to tests against wrinkling due to exposure to ultraviolet radiation. Specifically, the 10 women were divided into two groups. Each of the women in the two groups applied 1 g of the cream of Example 4 twice daily, once in the morning and once in the evening (a total application of 2 g daily), for a period of 14 days. Simultaneously, another group was established in which the women applied a cream containing no xanthone derivatives.

All of the women were exposed to ultraviolet radiation for one hour a day from an ultraviolet radiation apparatus for tanning (NEOTAN 888, Mrock Inc.). Before the first application of the cream of Example 4 and after 14 days of application of the lotion, measurements were taken from the facial skin of each subject. In particular, skin temperature was measured, water content of the corneum was measured using a skin surface hygrometer (SKICON 200, IBS Co., Ltd.), skin elasticity was measured using an elastometer (Cutometer), and the length of wrinkles per unit area was measured. Further, perspiration and sebum were collected to determine the contents of TNF alpha, an inflammatory cytokine, by immuno-enzyme technique (Amgen).

The results showed that, relative to the subjects using the control lotion, the skin of those women applying the cream of Example 4 exhibited an average decrease in temperature of skin of 0.44° C. In addition, the water content of the skin surface increased up to 159% with the use of the lotion of Example 4, relative to the water content of the skin of those using only the control lotion. Further, the elasticity of the skin of the women that applied the cream of Example 4 increased up to 166% relative to the women only applying the control lotion. In addition to the effects with respect to skin temperature, water content and elasticity, after 14 days of application, the skin of the women applying the cream of Example 4 exhibited a 55% reduction in length of wrinkles per unit area and a 31% reduction in the presence of TNF alpha in perspiration and sebum as compared to the same measurements taken before commencement of the application regimen.

### Example 6

### Antioxidant Composition Obtained Through Fermentation

Pericarps of mangosteens cultivated in Thailand were used as a raw material. First, mangosteen pericarps were washed with water, dried and ground by a grinder (DM-6, Nakayamalabo Corporation). 1 kg of the mangosteen grind was placed in a clean culture tank and 3 L of distilled water were added. 100 g of arginine manufactured by Ajinomoto Co., Ltd. were added. Soybeans produced in China were washed and warmed at 39° C. for 90 minutes, followed by grinding to obtain 1.5 kg of soybean grind, which was added to the reaction mixture. 10 g of lactic bacteria manufactured by Biofermin Pharmaceutical Co., Ltd. were added to the reaction mixture, and fermentation was carried out with agitation at 37° C. for 42 hours.

2 L of distilled water were added to the tank after fermentation to provide a fermented product, and 3 kg of coconut oil manufactured by Riken Vitamin Co., Ltd. were added to the fermented product. The combined oil and fermented product were agitated for 3 hours. The mixture was then left still to facilitate separation of the oil fraction from the aqueous fraction. The oil fraction containing the xanthone derivative was collected by decanting and filtered. In order to remove excess water, the collected oil fraction was fed into a TGD-250LF2 manufactured by TOYOGIKEN Co., Ltd. to obtain an extraction product in the form of an oil. This oil was designated the composition of Example 6.

### Example 7

### Preparation of Antioxidant Composition Through Reaction of a Mangosteen Grind with Arginine

2 kg of pericarps of mangosteen fruit produced in Thailand were washed thoroughly with distilled water and dried with a dryer. 1 kg of this mangosteen pericarp material was fed to a grinder (SANRIKI Almighty Crusher, Sanriki Seisakusho Co., Ltd.) and ground to obtain a mangosteen grind. A reaction mixture was formed by mixing 1 kg of the mangosteen grind in a clean pot with 9 L of distilled water and 230 g of arginine manufactured by Ajinomoto Co., Ltd. The reaction mixture was warmed at 60° C. and agitated for 12 hours at 65 rpm. The pot containing the warmed reaction mixture was cooled, and 2 kg of coconut oil manufactured by Riken Vitamin Co., Ltd. were added, agitated and collected as an extraction product in the form of an oil. This oil was designated the composition of Example 7.

### Example 8

### Preparation of Antioxidant Composition Through Reaction of Mangosteen, Peach Juice and a Transesterification Enzyme

2 kg of pericarps of mangosteen fruit produced in Thailand were washed thoroughly with distilled water and dried with a dryer (Type GZQ3, Nishimura Machine Works Co., Ltd.) to obtain a raw mangosteen material. 2 kg of this mangosteen material was fed to a grinder (SANRIKI Almighty Crusher, Sanriki Seisakusho Co., Ltd.) and ground to obtain a mangosteen grind. A reaction mixture was formed mixing 1 kg of the mangosteen grind in a clean pot, together with 10 L of distilled water and 1 kg of peach juice obtained by grinding peaches. This mixture was agitated, and 10 g of Lipase AY “Amano” 30G manufactured by Amano Enzyme Inc. were added to the reaction mixture, which was warmed at 37° C. and agitated for 10 hours at 70 rpm to provide a warmed reaction mixture. The pot containing the warmed reaction mixture was cooled with tap water, and 1 kg of coconut oil manufactured by Riken Vitamin Co., Ltd. was added, agitated, and collected by decanting and filtered to obtain an oily fraction. The fraction was dried by a vacuum dryer (Cleandry, AQM Kyushu Technos) to obtain an extraction product in the form of an oil. This oil was designated the composition of Example 8.

### Example 9

### Characterization of Composition of Example 6

The composition of Example 6 was characterized by HPLC and HPLC-MS. HPLC experiments were performed using a Waters Alliance System equipped with a vacuum degasser, quaternary solvent mixing, autosampler, and a Waters 996 diode array detector. UV spectra were collected across the range of 200-400 nm, extracting 254 nm for chromatograms. Waters Millennium32 software was utilized for instrument control, data collection, and data processing. The column was a Waters Nova-Pak C-8 (3.9×150 mm). The mobile phases consisted of A: 0.1% formic acid in water and B: methanol; optimal separations were achieved using a gradient of 65 to 90% B over 0-30 min at the flow rate of 1.0 mL/min. Injection volume for all the samples and standards was 10 μL. (Published reference: Edward B Walker, 2007, “HPLC analysis of selected xanthones in mangosteen fruit,” J. Sep. Sci. 30, 1229-1234.) HPLC-MS experiments were conducted on a Hewlett-Packard HPLC-MS using ESI positive mode with the same column, gradient, and flow rates.

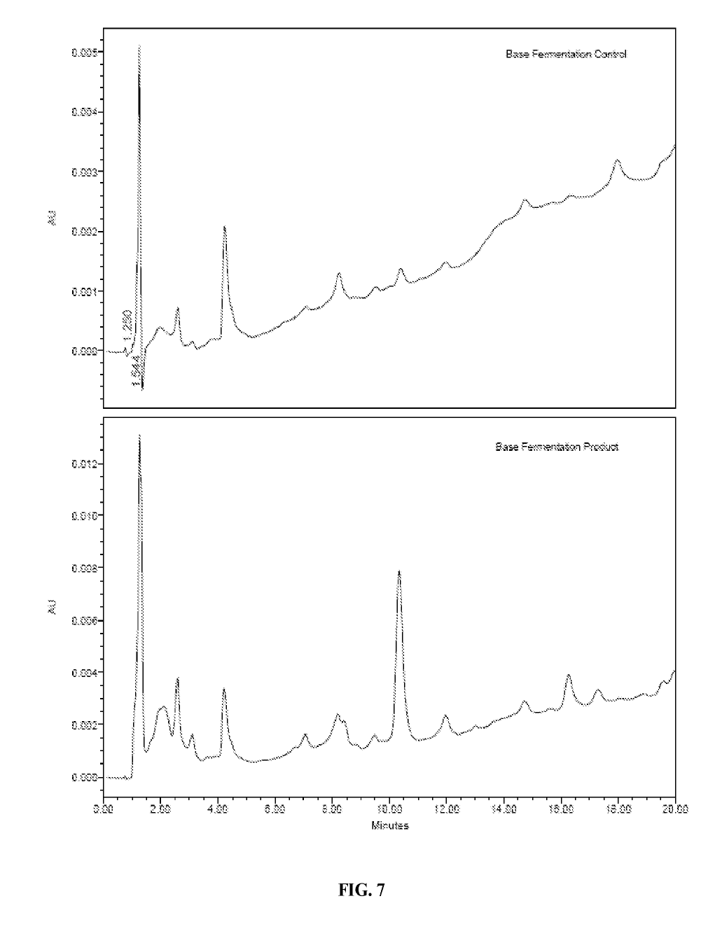

The composition of Example 6 was totally soluble in methanol, as was a control composition. The composition of Example 6 and a control composition were analyzed by HPLC-PDA and HPLC-MS. The composition of Example 6 exhibited two new peaks at 10.342 and 11.967 min detected by photodiode array (PDA) at 254 nm that were not present in the control composition (See FIG. 7). Neither of these peaks were not detected in HPLC-MS in the ESI positive mode.

### Example 10

### Determination of Antioxidant Activity

In this example, free radical-scavenging ability was determined as an indicator of antioxidant action. Specifically, free radicals produced by xanthine oxidase were determined by 1,1-diphenyl-2-picrylhydrayil (DPPH, Aldrich). First, xanthine oxidase (Aldrich) was dissolved in 0.01 M phosphate buffer. Hypoxanthine was added to this solution, and the solution was warmed at 37° C. to generate free radicals. To this solution, the composition of Example 6 was added. Further, 0.1 M DPPH solution was added to determine absorbance at 520 nm. Superoxide dismutase (SOD) activity was also determined using Electron Spin Resonance (ESR) in xanthine and xanthine-odixase system. In both cases, vitamin E was used as a positive control.

In the DPPH radical scavenging test, the composition of Example 6 at a dose of 0.1 mg/ml exhibited a free radical-scavenging ability of 24,454 units/g. For comparison, in the same test, the free radical-scavenging ability of VitaminE was 460 units/g. In addition, the SOD activity of the composition of Example 6 was observed to be 2,284,800 units/g, while the SOD activity of VitaminE was measured to be 90 units/g.

### Example 11

### Cosmetic Composition

A cosmetic composition including the composition obtained in Example 6 was produced. The cosmetic composition included 100 g of the composition of Example 6, and 10 g of grape juice extract manufactured by Kongo Yakuhin Co., Ltd. This mixture was mixed at a temperature of 30° C. over a period of 6 hours to obtain a composition. To 100 g of this composition, 0.2 g of sodium gluconate, 0.1 g of Lipase F-AP15 manufactured by Amano Enzyme Inc., and 1 g of polyethylene glycol monostearate were added, mixed, and warmed at 21° C. for 24 hours. The composition obtained by this process was cooled, and after cooling, 1 g of lipophilic glycerin monostearate, 2 g of horse oil ester and 3 g of oleic acid were mixed into the composition under heating to obtain a uniform composition. 2 g of propylene glycol, 0.1 g of α-tocopherol, and 70 g of distilled water were then added to the composition under heating and the resulting composition was cooled to obtain an emulsion. This emulsion was designated the lotion of Example 11.

### Example 12

### Testing for Sunburn-Improving Action of the Cosmetic Products

Using the lotion of Example 11, six women, ages ranging from 35 to 66, were subjected to improvement tests against sunburn due to exposure to ultraviolet radiation. The tests were carried out to measure improvement against wrinkling due to exposure to ultraviolet radiation. Specifically, six women were divided into two groups in which the women applied 1 g a day of the lotion of Example 11 to their faces for 14 days. Simultaneously, another group was established in which women applied a control lotion lacking the analyte of Example 6.

All of the women were exposed to ultraviolet radiation for one hour a day from an ultraviolet radiation apparatus for tanning (NEOTAN 888, Mrock Inc.). Before the first application of the lotion of Example 11 and 14 days after use of the lotion, measurements were taken from the facial skin of each subject. In particular, skin temperature was measured, water content of the corneum was measured using a skin surface hygrometer (SKICON 200, IBS Co., Ltd.), skin elasticity was measured using an elastometer (Cutometer), and the length of wrinkles per unit area was measured.

The results showed that, relative to the subjects using the control lotion, the skin of those women applying the lotion of Example 11 exhibited an average decrease in temperature of skin of 0.22° C. In addition, the water content of the skin surface increased up to 140% with the use of the lotion of Example 11, relative to the water content of the skin of those using only the control lotion. Further, the elasticity of the skin of the women that applied the lotion of Example 11 increased up to 156% relative to the women only applying the control lotion. Also, length of wrinkles measured in women applying the lotion of Example 11 decreased 33% relative to the women applying only the control lotion.

### Example 13

### Anti-Inflammatory Composition Obtained Through Fermentation

Pericarps of mangosteens cultivated in Thailand were used as a raw material. First, the mangosteen pericarps were washed with water and dried. They were then ground by a grinder (DM-6, Nakayamalabo Corporation). 1 kg of this mangosteen grind was placed in a clean culture tank and 3 L of distilled water were added. Soybeans produced in China were washed and warmed out at 39° C. for 90 minutes, followed by grinding to obtain 1.5 kg of soybean grind. 10 g of Bacillus natto manufactured by Nattomotohonpo were added. Fermentation was carried out with agitation at 37° C. for 40 hours. 3 L of distilled water were added to the tank after fermentation to provide a fermented product. 1 kg of coconut oil manufactured by Riken Vitamin Co., Ltd. was added to the fermented product and agitation was carried out for 3 hours for mixing. It was left still to collect a fat-soluble fraction separated by the coconut oil into the supernatant as a liquid. In order to remove water, it was placed in a TGD-250LF2 manufactured by TOYOGIKEN Co., Ltd. to obtain an anti-inflammatory composition as an oil. This oil was designated the extract of Example 13.

### Example 14

### Preparation of Anti-Inflammatory Composition Using Palmitic Acid Reaction Mixture

2 kg of pericarps of mangosteens produced in Thailand were washed thoroughly with distilled water and dried with a dryer. 1 kg of the dried pericarps was fed to a grinder (SANRIKI Almighty Crusher, Sanriki Seisakusho Co., Ltd.) and ground to obtain a mangosteen grind. 1 kg of the mangosteen grind was placed in a clean pot and 9 L of distilled water were added. 230 g of palmitic acid obtained from Ryo Shoku Ltd. were added and the reaction mixture was agitated. 10 g of Lipase AY “Amano” 30G manufactured by Amano Enzyme Inc. were added, and the reaction mixture was warmed at 33° C. and agitated for 9 hours at 65 rpm to provide a warmed liquid. The vessel containing the warmed reaction mixture was cooled, and 2 kg of coconut oil manufactured by Riken Vitamin Co., Ltd. were added, agitated, collected by decanting and filtered to provide an oily fraction. The oily fraction was dried by a vacuum dryer (Cleandry, AQM Kyushu Technos) to obtain, as an oily liquid, an anti-inflammatory composition. This liquid was designated the extract of Example 14.

### Example 15

### Preparation of Anti-Inflammatory Composition Using P-Coumaric Acid Reaction Mixture

2 kg of pericarps were obtained from mangosteens produced in Thailand, washed thoroughly with distilled water and dried with a dryer (Type GZQ3, Nishimura Machine Works Co., Ltd.) to obtain to a raw material mangosteen. 2 kg of this mangosteen were fed to a grinder (SANRIKI Almighty Crusher, Sanriki Seisakusho Co., Ltd.) and ground to obtain a mangosteen grind. 1 kg of the mangosteen grind was placed in a clean vessel, and 10 L of distilled water were added. 220 g of p-coumaric acid obtained from Anri Japan was added and the reaction mixture was agitated. 10 g of Lipase AY “Amano” 30G manufactured by Amano Enzyme Inc. were added, and the reaction mixture was warmed at 30° C. and agitated for 11 hours at 70 rpm to provide a warmed reaction mixture. The vessel containing the warmed reaction mixture was cooled with tap water, and 1 kg of coconut oil manufactured by Riken Vitamin Co., Ltd. was added, agitated, collected by decanting and filtered to provide an oily fraction. This oily fraction was dried by a vacuum dryer (Cleandry, AQM Kyushu Technos) to obtain, as an oily liquid, an anti-inflammatory composition. This liquid was designated the extract of Example 15.

### Example 16

### Preparation of Anti-Inflammatory Composition Using Adipic Acid Reaction Mixture

Pericarps were obtained from mangosteens produced in Thailand. 3 kg of the mangosteen pericarps were collected, washed thoroughly with distilled water and dried with a dryer to obtain mangosteen pericarps as a raw material. 2.2 kg of the mangosteen pericarps were fed to a grinder (SANRIKI Almighty Crusher, Sanriki Seisakusho Co., Ltd.) and ground to obtain a mangosteen pericarp grind. 1 kg of the mangosteen pericarp grind was placed in a clean vessel and 1.9 L of distilled water were added. 330 g of adipic acid manufactured by Anri Japan was added, and the reaction mixture was agitated. 20 g of Lipase AY “Amano” 30G manufactured by Amano Enzyme Inc. were added, warmed at 33° C. and agitated for 10 hours at 60 rpm to provide a warmed reaction mixture. The vessel containing the warmed reaction was cooled with tap water, and 1 kg of soy bean oil manufactured by Ajimonoto Co., Inc. was added, agitated, collected by decanting and filtered to provide an oily fraction. This oily fraction was dried by a vacuum dryer (Cleandry, AQM Kyushu Technos) to obtain, as an oily liquid, an anti-inflammatory composition. This liquid was designated the extract of Example 16.

### Example 17

### Preparation of Anti-Inflammatory Composition Using Xanthone-Carboxylic Reaction Mixture

2.9 kg of mangosteens cultivated in Thailand were purchased and pericarps were collected, washed thoroughly with distilled water and dried with a dryer (Type GZQ3, Nishimura Machine Works Co., Ltd.) to obtain mangosteen pericarps as a raw material. 2.5 kg of the mangosteen pericarps were fed to a grinder (SANRIKI Almighty Crusher, Sanriki Seisakusho Co., Ltd.) and ground to obtain a mangosteen pericarp grind. 1.1 kg of the mangosteen pericarp grind were placed in a clean vessel, and 10 L of distilled water were added. 350 g of xanthone-carboxylic acid manufactured by Anri Japan were added to the reaction mixture and agitated. 11 g of Lipase AY “Amano” 30G manufactured by Amano Enzyme Inc. were added, and the reaction mixture was warmed at 25° C. and agitated for 15 hours at 73 rpm to provide a warmed reaction mixture. The vessel containing the warmed liquid was cooled with tap water and then 1 kg of coconut oil manufactured by Riken Vitamin Co., Ltd. was added, agitated, collected by decanting and filtered to provide an oily fraction. This oily fraction was dried by a vacuum dryer (Cleandry, AQM Kyushu Technos) to obtain, as an oily liquid, an anti-inflammatory composition. This liquid was designated the extract of Example 17.

### Example 18

### Further Purification of Anti-Inflammatory Composition

100 g of the extract of Example 15 were suspended in 500 ml of ethanol and fed to a column filled with 900 g of Diaion manufactured by Mitsubishi Chemical Corporation. This was washed with 1000 ml of water containing 5% ethanol. Further, it was washed with 2000 ml of water containing 20% ethanol and eluted with 800 ml of water containing 70% ethanol and then a fraction of water containing 90% ethanol was collected.

This fraction was fed to a vacuum dryer to remove ethanol and water to obtain 15 g of an oily refined product of a xanthone derivative by a freeze-drying machine manufactured by Nippon Freeze Drying Co., Ltd. This product was designated the composition of Example 18.

### Example 19

### Characterization of Anti-Inflammatory Composition

The extract of Example 13 was characterized HPLC and HPLC-MS. HPLC experiments were performed using a Waters Alliance System equipped with a vacuum degasser, quaternary solvent mixing, autosampler, and a Waters 996 diode array detector. UV spectra were collected across the range of 200-400 nm, extracting 254 nm for chromatograms. Waters Millennium32 software was utilized for instrument control, data collection, and data processing. The column was a Waters Nova-Pak C-8 (3.9×150 mm). The mobile phases consisted of A: 0.1% formic acid in water and B: methanol; optimal separations were achieved using a gradient of 65 to 90% B over 0-30 min at the flow rate of 1.0 mL/min. Injection volume for all the samples and standards was 10 μL. (Published reference: Edward B Walker, 2007, “HPLC analysis of selected xanthones in mangosteen fruit,” J. Sep. Sci. 30, 1229-1234.) HPLC-MS experiments were conducted on a Hewlett-Packard HPLC-MS using ESI positive mode with the same column, gradient, and flow rates.

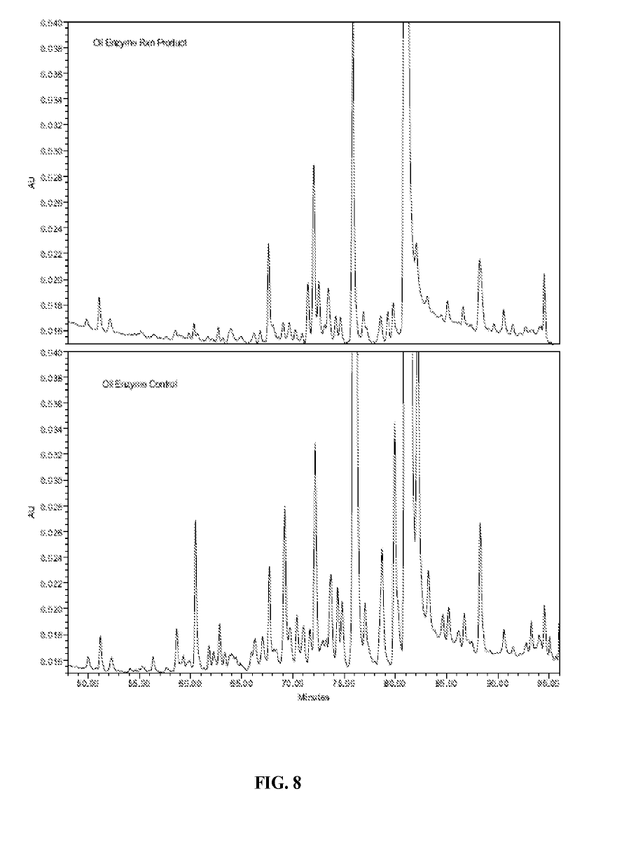

The extract of Example 13 was totally soluble in solvents ranging in polarity from very polar (e.g., water), to less polar, (e.g., alcohols), to extremely non-polar (e.g., hexane), while a control composition was not soluble in water or in lower-molecular weight alcohols, such as methanol or ethanol. When analyzed by HPLC-PDA and HPLC-MS, a number of peaks exhibited in the control composition were missing or reduced in the extract of Example 13 (See FIG. 8). Specifically, the chromatogram peak at 60.48 minutes is significantly reduced in total height and area. Similarly, the chromatogram peak at 69.06 minutes is significantly reduced. Moreover, relative to the control composition, the peak area and/or height of the peak at 78.60 minutes, the peak at 80.04 minutes, and a shoulder at 82.11 minutes are reduced. Even further, the extract of Example 13 exhibited a new chromatographic peak emerging at 79.20 minutes.

### Example 20

### Suppression of Inflammatory Cytokine Production by the Xanthone Derivatives

For testing, heparinized blood was sampled from five males, ages ranging from 25 to 55, and lymphocytes were cultured with an RPMI1640 medium (Nissui Pharmaceutical Co., Ltd.). 10,000 of the lymphocytes were seeded to a culture dish having a diameter of 35 mm and cultured under 5% carbon dioxide at 37° C. for 24 hours. 10 ng of lipopolysaccharide (LPS, Sigma-Aldrich) and the extract obtained in Example 13 were dissolved in dimethyl sulfoxide to form a test composition containing the extract of Example 13. The test composition included the extract of Example 13 at a concentration of 0.1 mg/ml. The test composition was added to the cell culture, and 24 hours after addition, culture supernatant was collected and quantified by absorptiometry using an ELISA kit (Amgen) using, as inflammatory cytokines, Interleukin-6 and TNF alpha. Dimethyl sulfoxide (Wako Pure Chemical Industries, Ltd.) without the extract of Example 13 was used as a control.

With respect to Interleukin-6, a result of 0.5% was obtained with addition of 0.1 mg/ml of the extract of Example 13, on the basis of 100% of the amount of the LPS control. That is, 99.5% of Interleukin-6 production was suppressed. With respect to TNF alpha, a result of 4% was obtained with addition of 1 ng of the extract of Example 13, on the basis of 100% of the amount of the solvent control. That is, 96% of TNF alpha production was suppressed.

1. A method for producing a composition for promoting collagen production, the method comprising:
providing a reaction mixture comprising a solvent, a mangosteen grind, a fermentation base, and one or more microorganisms that drive a fermentation process;
fermenting the reaction mixture to create a fermented product; and
extracting the xanthone derivative from the fermented product using an acidic aqueous solution.

2. A method according claim 1, wherein the acidic aqueous solution is selected from acetic acid, citric acid, malic acid, succinic acid, oxalic acid, hydrochloric acid, sulfuric acid, formic acid, propionic acid, butyric acid, alpha hydroxyl acid, phosphoric acid, and combinations of one or more thereof.

3. A food product comprising:
a composition for promoting collagen production, wherein the composition is obtained using a method according to claim 1; and
a juice extract included at about 0.001 parts to about 1 part by weight relative to 1 part by weight of the composition for promoting collagen production.

4. The food composition of claim 3, wherein the juice extract is derived from one or more fruits selected from apple, pear, cranberry, blueberry, strawberry, raspberry, cherry, tangerine, orange, fig, apricot, mango, melon, grape, loquat, banana, watermelon, durian and Cucurbitaceae.

5. A method of making the food composition of claim 4, the method comprising:
mixing the composition for promoting collagen production and juice extract; and
warming the combined composition for promoting collagen production and juice extract at a temperature selected from between about 25° C. and about 48° C., and for a period selected from between about 3 hours and about 48 hours.

6. A cosmetic product comprising:
a composition for promoting collagen production, wherein the composition is obtained using a method according to claim 1; and
a juice extract included at about 0.02 parts to about 2 parts by weight relative to 1 part by weight of the composition for promoting collagen production.

7. The cosmetic composition of claim 6, wherein the juice extract is derived from one or more fruits selected from apple, pear, cranberry, blueberry, strawberry, raspberry, cherry, tangerine, orange, fig, apricot, mango, melon, grape, loquat, banana, watermelon, durian and Cucurbitaceae.

8. A method of making the cosmetic composition of claim 6, the method comprising:
mixing the composition for promoting collagen production and juice extract; and
warming the combined composition for promoting collagen production and juice extract at a temperature selected from between about 25° C. and about 49° C., and for a period selected from between about 3 hours and about 60 hours.

9. A method for promoting collagen production in a subject, the method comprising, administering a composition for promoting collagen production, wherein the composition is obtained using a method according to claim 1.

10. A method for increasing the hydration, reducing the appearance of, or increasing the elasticity of the skin of a subject, the method comprising:
topically administering a cosmetic composition according to claim 6 over an affected area of the skin.

11. A method for promoting collagen production in a subject, the method comprising:
topically administering a cosmetic composition according to claim 6 to the subject.

12. A method for slowing the formation of, reducing the appearance of, reducing or alleviating wrinkles, spots, dullness or sagging of the skin in a subject, the method comprising:
topically administering a cosmetic composition according to claim 6 over an affected area of the skin.

13. A method for producing an antioxidant composition, the method comprising:
providing a reaction mixture comprising a solvent, mangosteen grind, and a lipase, wherein the pH of the reaction mixture is maintained at about 8 to about 9;
warming the reaction mixture to a temperature of about 25° C. to about 50° C.;
agitating the reaction mixture for a period of about 1 to about 48 hours;
mixing the fermented product with glycerin; and
collecting the glycerin fraction containing the antioxidant composition.

14. The method of claim 13, wherein providing the reaction mixture includes providing a reaction mixture comprising a solvent, mangosteen grind, peach juice, and a lipase, wherein the pH of the reaction mixture is maintained at about 8 to about 9.

15. A method for producing an antioxidant composition, the method comprising:
providing a reaction mixture comprising a solvent, mangosteen grind, and arginine;
warming the reaction mixture to a temperature of about 20° C. to about 60° C.;
agitating the reaction mixture for a period of about 1 to about 24 hours;
mixing the fermented product with ethanol; and
collecting the ethanol fraction containing the antioxidant composition.

16. A cosmetic composition comprising:
an antioxidant composition obtained according to claim 13; and
procyanidin included at about 0.05 parts to about 0.5 parts by weight relative to 1 part by weight of the antioxidant composition.

17. The cosmetic composition of claim 16, further comprising a juice extract included at about 0.05 parts to about 1 part by weight in relation to 1 part by weight of the antioxidant composition.

18. A method for reducing wrinkles on the skin of a subject, the method comprising:
topically administering a cosmetic composition according to claim 16 over an affected area of the skin.

19. A method for increasing the hydration of the skin of a subject, the method comprising:
topically administering a cosmetic composition according to claim 16 over an affected area of the skin.

20. A method for increasing the elasticity of the skin of a subject, the method comprising:
topically administering a cosmetic composition according to claim 16 over an affected area of the skin.

21. A method for producing an anti-inflammatory composition, the method comprising:
providing a mangosteen grind;
adding Bacillus natto, water, and ground soybeans to said mangosteen grind to provide a fermentation mixture;
fermenting said fermentation mixture to produce said anti-inflammatory composition;
adding a vegetable oil to the fermentation mixture;
agitating the combined vegetable oil and fermentation mixture; and
extracting an oil phase formed at least in part by the vegetable oil and containing said anti-inflammatory composition.

22. A method for producing an anti-inflammatory composition, the method comprising:
providing a mangosteen grind;
adding a lipase, a solvent, and an organic acid to said mangosteen grind to produce a reaction mixture;
warming and agitating said reaction mixture to produce said anti-inflammatory composition;
adding a vegetable oil to the reaction mixture;
agitating the combined vegetable oil and reaction mixture; and
extracting an oil phase formed at least in part by the vegetable oil and containing said anti-inflammatory composition.

23. A personal care product comprising:
an anti-inflammatory composition produced by a method selected from the group consisting of the method of claim 1 and the method of claim 2.

24. The personal care product of claim 23, further comprising a first vegetable oil containing one or more extracts selected from pine leaf extract, extract of persimmon leaf, chrysanthemum blossom extract, and lycium fruit extract, wherein the first vegetable oil is provided at about 0.05 parts to about 0.8 parts by weight in relation to 1 part by weight of the anti-inflammatory composition.

25. The personal care product according to claim 24, further comprising a second vegetable oil containing one or more extracts selected from pine leaf extract, extract of persimmon leaf, chrysanthemum blossom extract, and lycium fruit extract, wherein the second vegetable oil is provided at about 0.05 parts to about 0.8 parts by weight in relation to 1 part by weight of the anti-inflammatory composition.

26. A method for reducing an inflammatory response in a subject, the method comprising:
administering to the subject an anti-inflammatory composition produced by the method of claim 21.

27. A method for reducing an inflammatory response in a subject, the method comprising:
administering to the subject an anti-inflammatory composition produced by the method of claim 22.

28. A method for producing an anti-inflammatory composition, the method comprising:
providing a mangosteen grind;
adding Bacillus natto, water, and ground soybeans to said mangosteen grind to provide a fermentation mixture;
fermenting said fermentation mixture to produce said anti-inflammatory composition;
adding a vegetable oil to the fermentation mixture;
agitating the combined vegetable oil and fermentation mixture; and
extracting an oil phase formed at least in part by the vegetable oil.

29. A method for producing anti-inflammatory composition, the method comprising:
providing a mangosteen grind;
adding a lipase, a solvent, and an organic acid to said mangosteen grind to produce a reaction mixture;
warming and agitating said reaction mixture;
adding a vegetable oil to the reaction mixture;
agitating the composition formed by the vegetable oil and reaction mixture; and
extracting an oil phase formed at least in part by the vegetable oil.

30. A method for reducing an inflammatory response in a subject, the method comprising:
administering to the subject an anti-inflammatory composition produced by the method of claim 28.

31. A method for reducing an inflammatory response in a subject, the method comprising:
administering to the subject an anti-inflammatory composition produced by the method of claim 29.
